(19) United States
(12) Patent Application Publication
(10) Pub. No.: US 2025/0340835 A1
(43) Pub. Date: Nov. 6, 2025

(54) VASCULARIZED BRAIN ORGANOIDS AND METHODS OF MAKING AND USE

(71) Applicant: Children's Hospital Medical Center, Cincinnati, OH (US)

(72) Inventors: Ziyuan GUO, Union, KY (US); Lan DAO, Liberty Twp., OH (US)

(73) Assignee: Children's Hospital Medical Center, Cincinnati, OH (US)

(21) Appl. No.: 18/855,571

(22) PCT Filed: Apr. 21, 2023
(86) PCT No.: PCT/US2023/019465

§ 371 Date: Oct. 9, 2024

Related U.S. Application Data
(60) Provisional application No. 63/334,037, filed on Apr. 22, 2022.

(51) Int. Cl. C12N 5/0793; A61K 35/30; C12N 5/071

(52) U.S. Cl. CPC C12N 5/0619; A61K 35/30; C12N 5/0691; C12N 2500/84; C12N 2501/13; C12N 2501/999; C12N 2513/00; C12N 2533/90

(57) ABSTRACT

Disclosed herein are organoid models of the blood-brain barrier and methods for making the same from pluripotent stem cells. These organoids exhibit the complex organization of cells including neurons, endothelial cells, glial cells, and pericytes resembling the natural blood-brain barrier structure. These organoids may be used for studying the functions of the blood-brain barrier and cerebrovascular disorders.

### CROSS-REFERENCE TO RELATED APPLICATIONS

This application is a U.S. National Stage Application of International Application No. PCT/US2023/019465, filed Apr. 21, 2023, and claims priority to and the benefit of U.S. Provisional Patent Application No. 63/334,037, filed Apr. 22, 2022, the contents of which are incorporated herein by reference in their entireties.

### FIELD OF THE INVENTION

Aspects of the present disclosure relate generally to organoid compositions exhibiting a blood-brain barrier structure, and methods of making and use thereof.

### BACKGROUND

The blood-brain barrier (BBB) is a biological structure of great importance, as it selectively allows or prevents the crossing of molecules and other substances from the blood into the central nervous system. This offers protection against pathogens and immune-related components such as immune cells, antibodies, and cytokines to shield the central nervous system from the effects of peripheral immune function. However, this also results in difficulty in pharmaceutical molecules from accessing the central nervous system, including the brain, limiting their therapeutic efficacy unless specifically designed to cross the BBB. Accordingly, the BBB is an essential component to be considered during drug development, and there is a great need for robust in vitro and/or in vivo models to study the BBB.

### SUMMARY

Disclosed herein are methods for producing a vascularized brain organoid. In some embodiments, the methods comprise contacting a blood vessel organoid and a brain organoid; and culturing the blood vessel organoid and the brain organoid for a period of time until the blood vessel organoid and the brain organoid fuse together and blood vessels of the blood vessel organoid infiltrate the brain organoid, thereby forming the vascularized brain organoid. In some embodiments, neurons of the brain organoid innervate the blood vessels of the blood vessel organoid that have infiltrated the organoid. In some embodiments, the vascularized brain organoid comprises a blood-brain barrier that is formed between the brain organoid and all or a portion of the blood vessels of the blood vessel organoid that have infiltrated the brain organoid. In some embodiments, the blood-brain barrier comprises endothelial cells linked with tight junctions, astrocytes, and pericytes.

Also disclosed herein are the vascularized brain organoids produced according to the methods disclosed herein.

Also disclosed herein are methods of treating a cerebrovascular disease or a disease associated with blood-brain barrier dysfunction in a subject in need thereof. In some embodiments, the methods comprise administering any of the vascularized brain organoids disclosed herein, or a portion or fragment thereof, to the subject

Also disclosed herein are methods of screening. In some embodiments, the methods comprise contacting any of the vascularized brain organoids disclosed herein, or portions thereof, with a candidate compound or composition, and assessing the effects of the candidate compound or composition on the vascularized brain organoid or portions thereof. In some embodiments, the effect is transport of the candidate compound or composition across the blood-brain barrier of the organoid or portions thereof.

Exemplary embodiments of the present disclosure are provided in the following numbered embodiments:

1. A method for producing a vascularized brain organoid, comprising:
contacting a blood vessel organoid and a brain organoid; and
culturing the blood vessel organoid and the brain organoid for a period of time until the blood vessel organoid and the brain organoid fuse together and blood vessels of the blood vessel organoid infiltrate the brain organoid;
wherein neurons of the brain organoid innervate the blood vessels of the blood vessel organoid that have infiltrated the organoid;
thereby forming the vascularized brain organoid;
wherein the vascularized brain organoid comprises a blood-brain barrier that is formed between the brain organoid and all or a portion of the blood vessels of the blood vessel organoid that have infiltrated the brain organoid;
wherein the blood-brain barrier comprises endothelial cells linked with tight junctions, astrocytes, and pericytes.

2. The method of embodiment 1, wherein the endothelial cells express CD31, GLUT-1 and PDGFR-β; the tight junctions comprise claudin-5; the astrocytes express S100B, GFAP, and AQP4; and the pericytes express PDGFR-β, aSMA and NG2.

3. The method of embodiment 1 or 2, wherein the brain organoid is a forebrain organoid, a midbrain organoid, a hypothalamus organoid, a hippocampus organoid, a spinal cord organoid, or a striatal brain organoid.

4. The method of any one of embodiments 1-3, wherein the blood vessel organoid and the brain organoid are contacted and/or cultured in a basement membrane matrix or component thereof, optionally Matrigel.

5. The method of any one of embodiments 1-4, wherein the blood vessel organoid and the brain organoid are cultured for a period of time that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days.

6. The method of any one of embodiments 1-5, wherein the blood vessel organoid and the brain organoid are cultured with agitation, optionally shaking, for at least a portion of the period of time.

7. The method of any one of embodiments 1-6, wherein the blood vessel organoid and the brain organoid are cultured:
1) without agitation for 1, 2, 3, 4, 5, 6, 7, 8, 9, 10, 11, 12, 13, or 14 days, or any number of days within a range defined by any two of the aforementioned days; and subsequently
2) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days;
optionally wherein the agitation comprises shaking.

8. The method of any one of embodiments 1-7, wherein the blood vessel organoid and the brain organoid are cultured in a medium that promotes neuronal growth and/or vascular growth.

9. The method of any one of embodiments 1-8, wherein the blood vessel organoid and the brain organoid are cultured in a medium that comprises growth factors that promote neuronal growth and/or growth factors that promote vascular growth.

10. The method of embodiment 9, wherein the growth factors that promote neuronal growth comprise a cAMP pathway activator, ascorbic acid, BDNF, GDNF, or any combination thereof.

11. The method of embodiment 9 or 10, wherein the growth factors that promote vascular growth comprise growth serum, a VEGF pathway activator, an FGF pathway activator, or any combination thereof.

12. The method of any one of embodiments 1-11, wherein culturing the blood vessel organoid and the brain organoid comprises:
a) culturing the blood vessel organoid and the brain organoid without agitation for 4, 5, 6, 7, 8, 9, or 10 days, optionally 7 days; and
b) culturing the organoids of step a) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days;
wherein the organoids of step a) and step b) are cultured in a medium comprising growth factors that promote neuronal growth and/or growth factors that promote vascular growth;
optionally wherein the agitation comprises shaking;
optionally wherein the growth factors that promote neuronal growth comprise a cAMP pathway activator, ascorbic acid, BDNF, GDNF, or any combination thereof;
optionally wherein the growth factors that promote vascular growth comprise growth serum, a VEGF pathway activator, an FGF pathway activator, or any combination thereof;
optionally wherein the blood vessel organoid and the brain organoid are cultured in a basement membrane matrix or component thereof, optionally Matrigel.

13. The method of any one of embodiments 10-12, wherein the CAMP pathway activator is cAMP.

14. The method of any one of embodiments 10-13, wherein the cAMP pathway activator is provided at a concentration of, or of about, 10, 20, 30, 40, 50, 60, 70, 80, 90, 100, 110, 120, 130, 140, or 150 μM, or any concentration within a range defined by any two of the aforementioned concentrations, optionally 50 μM or about 50 μM.

15. The method of any one of embodiments 10-14, wherein the ascorbic acid is provided at a concentration of, or of about, 50, 60, 70, 80, 90, 100, 110, 120, 130, 140, 150, 160, 170, 180, 190, 200, 210, 220, 230, 240, 250, 260, 270, 280, 290, or 300 μM, or any concentration within a range defined by any two of the aforementioned concentrations, optionally 200 μM or about 200 μM.

16. The method of any one of embodiments 10-15, wherein the BDNF is provided at a concentration of, or of about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, optionally 20 ng/ml or about 20 ng/ml.

17. The method of any one of embodiments 10-16, wherein the GDNF is provided at a concentration of, or of about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, optionally 20 ng/mL or about 20 ng/mL.

18. The method of any one of embodiments 10-17, wherein the growth serum is fetal bovine serum (FBS).

19. The method of any one of embodiments 10-18, wherein the growth serum is provided at a concentration of or of about, 0.5%, 1%, 2%, 3%, 4%, 5%, 6%, 7%, 8%, 9%, 10%, 11%, 12%, 13%, 14%, 15%, 16%, 17%, 18%, 19%, or 20%, or any concentration within a range defined by any two of the aforementioned concentrations, optionally 15% or about 15%.

20. The method of any one of embodiments 10-19, wherein the VEGF pathway activator is VEGF.

21. The method of any one of embodiments 10-19, wherein the VEGF pathway activator is provided at a concentration of, or of about, 10, 20, 30, 40, 50, 60, 70, 80, 90, 100, 110, 120, 130, 140, or 150 ng/mL, or any concentration within a range defined by any two of the aforementioned concentrations, optionally 100 ng/ml or about 100 ng/ml.

22 The method of any one of embodiments 10-21, wherein the FGF pathway activator is FGF2.

23. The method of any one of embodiments 10-22, wherein the FGF pathway activator is provided at a concentration of, or of about, 10, 20, 30, 40, 50, 60, 70, 80, 90, 100, 110, 120, 130, 140, or 150 ng/ml, or any concentration within a range defined by any two of the aforementioned concentrations, optionally 100 ng/ml or about 100 ng/mL.

24 The method of any one of embodiments 1-23, wherein the blood vessel organoid and/or the brain organoid are derived from pluripotent stem cells, optionally embryonic stem cells or induced pluripotent stem cells.

25. The method of any one of embodiments 1-24, wherein the blood vessel organoid has been produced according to a method comprising:
contacting an angiogenic sprout with a Wnt pathway activator, an FGF pathway activator, a VEGF pathway activator, and optionally a growth serum, for a first period of time;
thereby forming the blood vessel organoid.

26. The method of embodiment 25, wherein the first period of time is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any number of days within a range defined by any two of the aforementioned number of days, optionally 5 days or at least 5 days.

27. The method of embodiment 25 or 26, wherein the angiogenic sprout has been produced according to a method comprising:
a) contacting pluripotent stem cells with a Wnt pathway activator and a BMP pathway activator for a second period of time to form vascular lineage cells; and
b) contacting the vascular lineage cells with a VEGF pathway activator and a second cAMP pathway activator for a third period of time;
thereby forming the angiogenic sprout.

28. The method of embodiment 27, wherein the second period of time is 1, 2, 3, 4, or 5 days, or any number of days within a range defined by any two of the aforementioned number of days, optionally 3 days.

29 The method of embodiment 27 or 28, wherein the third period of time is 1, 2, 3, or 4 days, or any number of days within a range defined by any two of the aforementioned number of days, optionally 2 days.

30. The method of any one of embodiments 25-29, wherein the BMP pathway activator is BMP4.

31. The method of any one of embodiments 25-30, wherein the BMP pathway activator is provided at a concentration of, or of about, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, optionally 30 ng/mL or about 30 ng/mL.

32. The method of any one of embodiments 25-31, wherein the Wnt pathway activator is CHIR99021.

33. The method of any one of embodiments 25-32, wherein the Wnt pathway activator is provided at a concentration of, or of about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, optionally 4 μM or about 4 μM, or optionally 12 μM or about 12 μM.

34 The method of any one of embodiments 25-33, wherein the second cAMP pathway activator is forskolin.

35. The method of any one of embodiments 25-34, wherein the second cAMP pathway activator is provided at a concentration of, or of about, 0.5, 1, 1.5, 2, 2.5, 3, 3.5, or 4 μM, or any concentration within a range defined by any two of the aforementioned concentrations, optionally 2 μM or about 2 μM.

36. The method of any one of embodiments 25-35, wherein the blood vessel organoid differs from a blood vessel organoid that has been produced without contacting the angiogenic sprout with the Wnt pathway activator by having increased expression of blood-brain barrier-specific endothelial markers, optionally GLUT-1 and ZO-1.

37. The method of any one of embodiments 1-36, wherein the brain organoid has been contacted with LIF and growth serum to induce astrocyte formation in the brain organoid.

38. The method of any one of embodiments 1-37, wherein the brain organoid has been produced according to a method comprising:
a) contacting pluripotent stem cells with a BMP pathway inhibitor, a TGF-beta pathway inhibitor, and a Wnt pathway inhibitor for a first period of time to form neuroectoderm cells;
b) contacting the neuroectoderm cells of step a) with a second TGF-beta pathway inhibitor, and a Wnt pathway activator for a second period of time to form neuroepithelium cells;
c) contacting the neuroepithelium cells of step b) with insulin for a third period of time to form cerebral tissue organoids; and
d) contacting the cerebral tissue organoid of step c) with GDNF, BDNF, ascorbic acid, and a cAMP pathway activator for a fourth period of time to form the brain organoid;
optionally wherein the cerebral tissue organoid is further contacted with LIF and growth serum for a portion of the fourth period of time to induce astrocyte proliferation in the brain organoid.

39 The method of embodiment 38, wherein the first period of time is 1, 2, 3, 4, 5, 6, 7, 8, 9, 10, 11, 12, 13, or 14 days, or any number of days within a range defined by any two of the aforementioned number of days, optionally 7 days.

40. The method of embodiment 38 or 39, wherein the second period of time is 1, 2, 3, 4, 5, 6, 7, 8, 9, 10, 11, 12, 13, or 14 days, or any number of days within a range defined by any two of the aforementioned number of days, optionally 7 days.

41. The method of any one of embodiments 38-40, wherein the third period of time is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days, optionally 56 days.

42. The method of any one of embodiments 38-41, wherein the fourth period of time is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days.

43. The method of embodiment 42, wherein the portion of the fourth period of time is 7, 8, 9, 10, 11, 12, 13, 14, 15, 16, 17, 17, 18, 19, 20, or 21 days, or any number of days within a range defined by any two of the aforementioned number of days, optionally 14 days, optionally wherein the portion of the fourth period of time is at the beginning of the fourth period of time.

44 The method of embodiment 38-43, wherein the BMP pathway inhibitor is LDN-193189.

45. The method of any one of embodiments 38-44, wherein the BMP pathway inhibitor is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

46. The method of any one of embodiments 38-45, wherein the TGF-beta pathway inhibitor and the second TGF-beta pathway inhibitor is the same or different.

47. The method of any one of embodiments 38-46, wherein the TGF-beta pathway inhibitor is A83-01.

48 The method of any one of embodiments 38-47, wherein the TGF-beta pathway inhibitor is provided at a concentration of, or of about, 0.5, 1, 1.5, 2, 2.5, 3, 3.5, or 4 μM, or any concentration within a range defined by any two of the aforementioned concentrations, optionally 2 μM or about 2 μM.

49. The method of any one of embodiments 38-48, wherein the second TGF-beta pathway inhibitor is SB-431542.

50 The method of any one of embodiments 38-49, wherein the second TGF-beta pathway inhibitor is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

51. The method of any one of embodiments 38-50, wherein the Wnt pathway inhibitor is IWR-1.

52. The method of any one of embodiments 38-51, wherein the Wnt pathway inhibitor is provided at a concentration of, or of about, 0.5, 1, 1.5, 2, 2.5, 3, 3.5, 4, 4.5, or 5 μM, or any concentration within a range defined by any two of the aforementioned concentrations, optionally 3 μM or about 3 μM.

53. The method of any one of embodiments 38-52, wherein the Wnt pathway activator is CHIR99021.

54. The method of any one of embodiments 38-53, wherein the Wnt pathway activator is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

55. The method of any one of embodiments 38-54, wherein the insulin is provided at a concentration of, or of about, 0.5, 1, 1.5, 2, 2.5, 3, 3.5, 4, 4.5, or 5 μg/mL, or any concentration within a range defined by any two of the aforementioned concentrations, optionally 2.5 μg/mL or about 2.5 ug/mL.

56. The method of any one of embodiments 38-55, wherein the LIF is provided at a concentration of, or of about, 1, 2, 3, 4, 5, 6, 7, 8, 9, 10, 11, 12, 13, 14, 15, 16, 17, 18, 19, or 20 mg/mL, optionally 10 mg/mL or about 10 mg/mL.

57. The method of any one of embodiments 38-56, wherein the brain organoid comprises astrocytes that express S100B, GFAP, and AQP4.

58. The method of any one of embodiments 1-57, wherein the blood vessel organoid and/or the brain organoid are human.

59. The method of any one of embodiments 1-58, wherein the blood vessel organoid and/or the brain organoid have been derived from a subject, optionally a human subject.

60. The method of embodiment 59, wherein the subject comprises a cerebrovascular disease or a disease associated with blood-brain barrier dysfunction, optionally wherein the cerebrovascular disease or disease associated with blood-brain barrier dysfunction comprises cerebral cavernous malformation, Alzheimer's disease, or amyotrophic lateral sclerosis.

61. The vascularized brain organoid produced by the method of any one of embodiments 1-60.

62. A method of treating a cerebrovascular disease or a disease associated with blood-brain barrier dysfunction in a subject in need thereof, comprising administering the vascularized brain organoid of embodiment 61, or a portion or fragment thereof, to the subject.

63. A method of screening, comprising contacting the vascularized brain organoid of embodiment 61, or portions thereof, with a candidate compound or composition, and assessing the effects of the candidate compound or composition on the vascularized brain organoid or portions thereof.

64. The method of embodiment 63, wherein the effects comprises transport of the candidate compound or composition across the blood-brain barrier of the organoid or portions thereof.

65. The method of embodiment 63 or 64, wherein the vascularized brain organoid is a model for a cerebrovascular disease or a disease associated with blood-brain barrier dysfunction, and assessing the effects of the candidate compound or composition on the vascularized organoid comprises assessing the effects of the candidate compound or composition on the cerebrovascular disease or the disease associated with blood-brain barrier dysfunction.

66. The method of any one of embodiments 63-65, wherein the vascularized brain organoid has been produced from cells derived from a subject, optionally wherein the cells derived from the subject are induced pluripotent stem cells.

67. The method of embodiment 66, wherein the subject has or is disposed to develop the cerebrovascular disease or the disease associated with blood-brain barrier dysfunction.

Further exemplary embodiments of the present disclosure are provided in the following numbered embodiments:

1. A method for producing a vascularized brain organoid, comprising:
contacting a blood vessel organoid and a brain organoid; and
culturing the blood vessel organoid and the brain organoid for a period of time until the blood vessel organoid and the brain organoid fuse together and blood vessels of the blood vessel organoid infiltrate the brain organoid;
wherein neurons of the brain organoid innervate the blood vessels of the blood vessel organoid that have infiltrated the organoid;
thereby forming the vascularized brain organoid;
wherein the vascularized brain organoid comprises a blood-brain barrier that is formed between the brain organoid and all or a portion of the blood vessels of the blood vessel organoid that have infiltrated the brain organoid;
wherein the blood-brain barrier comprises endothelial cells linked with tight junctions, astrocytes, and pericytes.

2. The method of embodiment 1, wherein the endothelial cells express CD31, GLUT-1 and PDGFR-β; the tight junctions comprise claudin-5, ZO-1 and cadherin 5; the astrocytes express S100B, GFAP, and AQP4; and the pericytes express PDGFR-β, aSMA and NG2.

3. The method of embodiment 1 or 2, wherein the endothelial cells form a continuous basement membrane and express collagen IV.

4. The method of any of embodiments 1-3, wherein the vascularized brain organoid comprises cells selected from the group consisting of neural progenitors, proliferative astrocytes, GABAergic neurons, glutamatergic neurons, proliferative cells, brain vascular endothelial cells, vascular leptomeningeal cells, perivascular adipocytes, and tendon cells.

5. The method of any of embodiments 1-3, wherein the vascularized brain organoid comprises cells selected from the group consisting of neural progenitor cells, GABAergic neurons, glutamatergic neurons, proliferative astrocytes, proliferative GABAergic neurons, mesenchymal stem cells, endothelial cells, pericytes, vascular smooth muscle cells, fibroblast, and proliferative cells.

6. The method of embodiment 4 or 5, wherein the cells of the vascular brain organoid are identified by cell-type specific gene expression markers.

7. The method of any of embodiments 1-6, wherein the blood vessels comprise capillaries.

8. The method of embodiment 7, wherein the capillaries are ensheathed by pericytes and end-feet of the astrocytes.

9. The method of any of embodiments 1-8, wherein the brain organoid is a forebrain organoid, a midbrain organoid, a hypothalamus organoid, a hippocampus organoid, a spinal cord organoid, or a striatal brain organoid.

10. The method of any one of embodiments 1-9, wherein the blood vessel organoid and the brain organoid are contacted and/or cultured in a basement membrane matrix or component thereof, optionally Matrigel.

11. The method of any one of embodiments 1-10, wherein the blood vessel organoid and the brain organoid are cultured for a period of time that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days.

12. The method of any one of embodiments 1-11, wherein the blood vessel organoid and the brain organoid are cultured with agitation, optionally shaking, for at least a portion of the period of time.

13. The method of any one of embodiments 1-12, wherein the blood vessel organoid and the brain organoid are cultured:
1) without agitation for 1, 2, 3, 4, 5, 6, 7, 8, 9, 10, 11, 12, 13, or 14 days, or any number of days within a range defined by any two of the aforementioned days; and subsequently
2) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days;
optionally wherein the agitation comprises shaking.

14. The method of any one of embodiments 1-13, wherein the blood vessel organoid and the brain organoid are cultured in a medium that promotes neuronal growth and/or vascular growth.

15. The method of any one of embodiments 1-14, wherein the blood vessel organoid and the brain organoid are cultured in a medium that comprises growth factors that promote neuronal growth and/or growth factors that promote vascular growth.

16. The method of embodiment 15, wherein the growth factors that promote neuronal growth comprise a cAMP pathway activator, ascorbic acid, BDNF, GDNF, or any combination thereof.

17. The method of embodiment 15 or 16, wherein the growth factors that promote vascular growth comprise growth serum, a VEGF pathway activator, an FGF pathway activator, or any combination thereof.

18. The method of any one of embodiments 1-17, wherein culturing the blood vessel organoid and the brain organoid comprises:
a) culturing the blood vessel organoid and the brain organoid without agitation for 4, 5, 6, 7, 8, 9, or 10 days, optionally 7 days; and
b) culturing the organoids of step a) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days;
wherein the organoids of step a) and step b) are cultured in a medium comprising growth factors that promote neuronal growth and/or growth factors that promote vascular growth;
optionally wherein the agitation comprises shaking;
optionally wherein the growth factors that promote neuronal growth comprise a cAMP pathway activator, ascorbic acid, BDNF, GDNF, or any combination thereof;
optionally wherein the growth factors that promote vascular growth comprise growth serum, a VEGF pathway activator, an FGF pathway activator, or any combination thereof;
optionally wherein the blood vessel organoid and the brain organoid are cultured in a basement membrane matrix or component thereof, optionally Matrigel.

19. The method of any one of embodiments 16-18, wherein the CAMP pathway activator is cAMP.

20. The method of any one of embodiments 16-19, wherein the CAMP pathway activator is provided at a concentration of, or of about, 10, 20, 30, 40, 50, 60, 70, 80, 90, 100, 110, 120, 130, 140, or 150 μM, or any concentration within a range defined by any two of the aforementioned concentrations, optionally 50 μM or about 50 μM.

21. The method of any one of embodiments 16-20, wherein the ascorbic acid is provided at a concentration of, or of about, 50, 60, 70, 80, 90, 100, 110, 120, 130, 140, 150, 160, 170, 180, 190, 200, 210, 220, 230, 240, 250, 260, 270, 280, 290, or 300 μM, or any concentration within a range defined by any two of the aforementioned concentrations, optionally 200 μM or about 200 μM.

22. The method of any one of embodiments 16-21, wherein the BDNF is provided at a concentration of, or of about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, optionally 20 ng/ml or about 20 ng/mL.

23. The method of any one of embodiments 16-22, wherein the GDNF is provided at a concentration of, or of about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, optionally 20 ng/ml or about 20 ng/mL.

24. The method of any one of embodiments 17-23, wherein the growth serum is fetal bovine serum (FBS).

25. The method of any one of embodiments 17-24, wherein the growth serum is provided at a concentration of or of about, 0.5%, 1%, 2%, 3%, 4%, 5%, 6%, 7%, 8%, 9%, 10%, 11%, 12%, 13%, 14%, 15%, 16%, 17%, 18%, 19%, or 20%, or any concentration within a range defined by any two of the aforementioned concentrations, optionally 15% or about 15%.

26. The method of any one of embodiments 17-25, wherein the VEGF pathway activator is VEGF.

27. The method of any one of embodiments 17-26, wherein the VEGF pathway activator is provided at a concentration of, or of about, 10, 20, 30, 40, 50, 60, 70, 80, 90, 100, 110, 120, 130, 140, or 150 ng/mL, or any concentration within a range defined by any two of the aforementioned concentrations, optionally 100 ng/ml or about 100 ng/mL.

28. The method of any one of embodiments 17-27, wherein the FGF pathway activator is FGF2.

29. The method of any one of embodiments 17-28, wherein the FGF pathway activator is provided at a concentration of, or of about, 10, 20, 30, 40, 50, 60, 70, 80, 90, 100, 110, 120, 130, 140, or 150 ng/mL, or any concentration within a range defined by any two of the aforementioned concentrations, optionally 100 ng/ml or about 100 ng/mL.

30. The method of any one of embodiments 1-29, wherein the blood vessel organoid and/or the brain organoid are derived from pluripotent stem cells, optionally embryonic stem cells or induced pluripotent stem cells.

31. The method of any one of embodiments 1-30, wherein the blood vessel organoid has been produced according to a method comprising:
contacting an angiogenic sprout with an FGF pathway activator, a VEGF pathway activator, optionally a Wnt pathway activator, and optionally a growth serum, for a first period of time;
thereby forming the blood vessel organoid.

32. The method of embodiment 31, wherein the first period of time is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any number of days within a range defined by any two of the aforementioned number of days, optionally 5 days or at least 5 days.

33. The method of embodiment 31 or 32, wherein the angiogenic sprout has been produced according to a method comprising:
a) contacting pluripotent stem cells with a Wnt pathway activator and a BMP pathway activator for a second period of time to form vascular lineage cells; and
b) contacting the vascular lineage cells with a VEGF pathway activator and a second cAMP pathway activator for a third period of time;
thereby forming the angiogenic sprout.

34. The method of embodiment 33, wherein the second period of time is 1, 2, 3, 4, or 5 days, or any number of days within a range defined by any two of the aforementioned number of days, optionally 3 days.

35. The method of embodiment 33 or 34, wherein the third period of time is 1, 2, 3, or 4 days, or any number of days within a range defined by any two of the aforementioned number of days, optionally 2 days.

36. The method of any one of embodiments 33-35, wherein the BMP pathway activator is BMP4.

37. The method of any one of embodiments 33-36, wherein the BMP pathway activator is provided at a concentration of, or of about, 10, 15, 20, 25, 30, 35, 40, 45, 50, 55, 60, 65, 70, 75, 80, 85, 90, 95, or 100 ng/ml, or any concentration within a range defined by any two of the aforementioned concentrations, optionally 30 ng/ml or about 30 ng/mL.

38. The method of any one of embodiments 31-37, wherein the Wnt pathway activator is CHIR99021.

39. The method of any one of embodiments 31-38, wherein the Wnt pathway activator is provided at a concentration of, or of about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, optionally 4 μM or about 4 μM, or optionally 12 μM or about 12 μM.

40. The method of any one of embodiments 33-39, wherein the second cAMP pathway activator is forskolin.

41. The method of any one of embodiments 33-40, wherein the second cAMP pathway activator is provided at a concentration of, or of about, 0.5, 1, 1.5, 2, 2.5, 3, 3.5, or 4 μM, or any concentration within a range defined by any two of the aforementioned concentrations, optionally 2 μM or about 2 μM.

42. The method of any one of embodiments 31-41, wherein the blood vessel organoid differs from a blood vessel organoid that has been produced without contacting the angiogenic sprout with the Wnt pathway activator by having increased expression of blood-brain barrier-specific endothelial markers, optionally GLUT-1 and ZO-1.

43. The method of any one of embodiments 1-42, wherein the brain organoid has been contacted with LIF and growth serum to induce astrocyte formation in the brain organoid.

44. The method of any one of embodiments 1-43, wherein the brain organoid has been produced according to a method comprising:
a) contacting pluripotent stem cells with a BMP pathway inhibitor, a TGF-beta pathway inhibitor, and a Wnt pathway inhibitor for a first period of time to form neuroectoderm cells;
b) contacting the neuroectoderm cells of step a) with a second TGF-beta pathway inhibitor, and a Wnt pathway activator for a second period of time to form neuroepithelium cells;
c) contacting the neuroepithelium cells of step b) with insulin for a third period of time to form cerebral tissue organoids; and
d) contacting the cerebral tissue organoid of step c) with GDNF, BDNF, ascorbic acid, and a cAMP pathway activator for a fourth period of time to form the brain organoid;
optionally wherein the cerebral tissue organoid is further contacted with LIF and growth serum for a portion of the fourth period of time to induce astrocyte proliferation in the brain organoid.

45. The method of embodiment 44, wherein the first period of time is 1, 2, 3, 4, 5, 6, 7, 8, 9, 10, 11, 12, 13, or 14 days, or any number of days within a range defined by any two of the aforementioned number of days, optionally 7 days.

46. The method of embodiment 44 or 45, wherein the second period of time is 1, 2, 3, 4, 5, 6, 7, 8, 9, 10, 11, 12, 13, or 14 days, or any number of days within a range defined by any two of the aforementioned number of days, optionally 7 days.

47. The method of any one of embodiments 44-46, wherein the third period of time is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days, optionally 56 days.

48. The method of any one of embodiments 44-47, wherein the fourth period of time is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days.

49. The method of any one of embodiments 44-48, wherein the portion of the fourth period of time is 7, 8, 9, 10, 11, 12, 13, 14, 15, 16, 17, 17, 18, 19, 20, or 21 days, or any number of days within a range defined by any two of the aforementioned number of days, optionally 14 days, optionally wherein the portion of the fourth period of time is at the beginning of the fourth period of time.

50. The method of any of embodiments 44-49, wherein the BMP pathway inhibitor is LDN-193189.

51. The method of any one of embodiments 44-50, wherein the BMP pathway inhibitor is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

52. The method of any one of embodiments 44-51, wherein the TGF-beta pathway inhibitor and the second TGF-beta pathway inhibitor is the same or different.

53. The method of any one of embodiments 44-52, wherein the TGF-beta pathway inhibitor is A83-01.

54. The method of any one of embodiments 44-53, wherein the TGF-beta pathway inhibitor is provided at a concentration of, or of about, 0.5, 1, 1.5, 2, 2.5, 3, 3.5, or 4 μM, or any concentration within a range defined by any two of the aforementioned concentrations, optionally 2 μM or about 2 μM.

55. The method of any one of embodiments 44-54, wherein the second TGF-beta pathway inhibitor is SB-431542.

56. The method of any one of embodiments 44-55, wherein the second TGF-beta pathway inhibitor is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

57. The method of any one of embodiments 44-56, wherein the Wnt pathway inhibitor is IWR-1.

58. The method of any one of embodiments 44-57, wherein the Wnt pathway inhibitor is provided at a concentration of, or of about, 0.5, 1, 1.5, 2, 2.5, 3, 3.5, 4, 4.5, or 5 μM, or any concentration within a range defined by any two of the aforementioned concentrations, optionally 3 μM or about 3 μM.

59. The method of any one of embodiments 44-58, wherein the Wnt pathway activator is CHIR99021.

60. The method of any one of embodiments 44-59, wherein the Wnt pathway activator is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

61. The method of any one of embodiments 44-60, wherein the insulin is provided at a concentration of, or of about, 0.5, 1, 1.5, 2, 2.5, 3, 3.5, 4, 4.5, or 5 μg/mL, or any concentration within a range defined by any two of the aforementioned concentrations, optionally 2.5 μg/mL or about 2.5 ug/mL.

62. The method of any one of embodiments 44-61, wherein the LIF is provided at a concentration of, or of about, 1, 2, 3, 4, 5, 6, 7, 8, 9, 10, 11, 12, 13, 14, 15, 16, 17, 18, 19, or 20 mg/mL, optionally 10 mg/mL or about 10 mg/mL.

63. The method of any one of embodiments 44-62, wherein the brain organoid comprises astrocytes that express S100B, GFAP, and AQP4.

64. The method of any one of embodiments 1-63, wherein the blood vessel organoid and/or the brain organoid are human.

65. The method of any one of embodiments 1-64, wherein the blood vessel organoid and/or the brain organoid have been derived from a subject, optionally a human subject.

66. The method of embodiment 65, wherein the subject comprises a cerebrovascular disease or a disease associated with blood-brain barrier dysfunction, optionally wherein the cerebrovascular disease or disease associated with blood-brain barrier dysfunction comprises cerebral cavernous malformation, Alzheimer's disease, or amyotrophic lateral sclerosis.

67. The vascularized brain organoid produced by the method of any one of embodiments 1-66.

68. A vascularized brain organoid comprising endothelial cells linked with tight junctions, astrocytes, and pericytes.

69. The vascularized brain organoid of embodiment 68, wherein the endothelial cells express CD31, GLUT-1 and PDGFR-β; the tight junctions comprise claudin-5, ZO-1 and cadherin 5; the astrocytes express S100B, GFAP, and AQP4; and the pericytes express PDGFR-β, aSMA and NG2

70. The vascularized brain organoid of embodiment 68 or 69, wherein the endothelial cells form a continuous basement membrane and express collagen IV.

71. The vascularized brain organoid of any of embodiments 68-70 comprising cells selected from the group consisting of neural progenitors, proliferative astrocytes, GABAergic neurons, glutamatergic neurons, proliferative cells, brain vascular endothelial cells, vascular leptomeningeal cells, perivascular adipocytes, and tendon cells.

72. The vascularized brain organoid of any of embodiments 68-71 comprising cells selected from the group consisting of neural progenitor cells, GABAergic neurons, glutamatergic neurons, proliferative astrocytes, proliferative GABAergic neurons, mesenchymal stem cells, endothelial cells, pericytes, vascular smooth muscle cells, fibroblast, and proliferative cells.

73. The vascularized brain organoid of embodiments 71 or 72, wherein the cells are identified by cell-type specific gene expression markers.

74. The vascularized brain organoid of any of embodiments 68-73, wherein the blood vessels comprise capillaries.

75. The vascularized brain organoid of any of embodiments 68-74, wherein the capillaries are ensheathed by pericytes and end-feet of the astrocytes.

76. A method of treating a cerebrovascular disease or a disease associated with blood-brain barrier dysfunction in a subject in need thereof, comprising administering the vascularized brain organoid of any of embodiments 67-75, or a portion or fragment thereof, to the subject.

77. A method of screening, comprising contacting the vascularized brain organoid of any of embodiments 67-75, or portions thereof, with a candidate compound or composition, and assessing the effects of the candidate compound or composition on the vascularized brain organoid or portions thereof.

78. The method of embodiment 77, wherein the effects comprises transport of the candidate compound or composition across the blood-brain barrier of the organoid or portions thereof.

79. The method of embodiment 77 or 78, wherein the vascularized brain organoid is a model for a cerebrovascular disease or a disease associated with blood-brain barrier dysfunction, and assessing the effects of the candidate compound or composition on the vascularized organoid comprises assessing the effects of the candidate compound or composition on the cerebrovascular disease or the disease associated with blood-brain barrier dysfunction.

80. The method of any one of embodiments 77-79, wherein the vascularized brain organoid has been produced from cells derived from a subject, optionally wherein the cells derived from the subject are induced pluripotent stem cells.

81. The method of embodiment 80, wherein the subject has or is disposed to develop the cerebrovascular disease or the disease associated with blood-brain barrier dysfunction.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A depicts an embodiment of a schematic for forming blood vessel (vascular) organoids from pluripotent stem cells.

FIG. 1B depicts an embodiment of brightfield and fluorescent images of blood vessel organoids differentiated from pluripotent stem cells according to the exemplary schematic of FIG. 1A. The fluorescent images show cells expressing GFP, which the initial pluripotent stem cells were engineered to express.

FIGS. 1C-1D depict an embodiment of fluorescent images of blood vessel organoids differentiated from pluripotent stem cells according to the exemplary schematic of FIG. 1A. The fluorescent images show that the cells of the blood vessel organoids express GFP (which the initial pluripotent stem cells were engineered to express), platelet endothelial cell adhesion molecule (PECAM-1; CD31) and platelet-derived growth factor receptor beta (PDGFR-β), which are markers for early endothelial and pericyte progenitor cells. The fluorescent images of FIG. 1D show tube structures with a lumen, indicating the generation of 3D self-organized blood vessel organoids.

FIG. 2A depicts an embodiment of a schematic for forming a dorsal forebrain organoid, which is considered a type of cortical organoid, from pluripotent stem cells.

FIG. 2B depicts an embodiment of brightfield and fluorescent images of forebrain organoids differentiated from pluripotent stem cells according to the exemplary schematic of FIG. 2A. The fluorescent images show that the cells of early (Day 26) forebrain organoids express the neuronal cell marker class III beta-tubulin (Tuj1) and neural stem cell marker SRY-Box transcription factor (Sox2). The cells of later (Day 61) forebrain organoids express the neuronal cell markers B-cell lymphoma/leukemia 11B (BCL11B; Ctip1) and T-Box brain transcription factor 1 (Tbr1).

FIGS. 2C-2D depict an embodiment of fluorescent images of forebrain organoids differentiated from pluripotent stem cells and cultured to induced astrocyte formation. The fluorescent images show that the forebrain organoids contain cells that are positive for astrocyte markers $100 calcium binding protein B (S100B), and glial fibrillary acidic protein (GFAP).

FIG. 3A depicts an embodiment of a schematic for fusing a blood vessel organoid (VO) and a forebrain organoid (FBO) in culture to form a fused vascularized forebrain organoid (fvFBO). Alternative cortical organoids may be used instead of a forebrain organoid.

FIG. 3B depicts an embodiment of a schematic for generating vascularized brain organoids from human pluripotent stem cells (hPSCs). Cerebral organoids and blood vessel organoids are generated separately and then assembled to mimic neurovascular co-development.

FIG. 3C depicts an embodiment of a schematic of a vascularized brain organoid and its resemblance to the in vivo human BBB-like structure with endothelial cells of the capillary wall connected through tight junctions, astrocytic end-feet and pericytes ensheathing, as well as neuron innervation.

FIG. 3D depicts an embodiment of an exemplary protocol for culturing a blood vessel organoid and forebrain organoid together to form a fused vascularized forebrain organoid (fvFBO). The blood vessel organoid and forebrain organoid are directly contacted to fuse together in culture containing growth factors that promote both neuronal and vascular growth. Also depicted are fluorescent images showing the fused vascularized forebrain organoid with endothelial cell infiltration into the brain organoid. Alternative cortical organoids may be used instead of a forebrain organoid.

FIG. 3E depicts an embodiment of fluorescent images showing the integration of the blood vessel organoid and forebrain organoid in the fused vascularized forebrain organoid after 14 and 21 days of culture. As culture time progresses, endothelial cells (which are labeled with GFP) progressively infiltrate the forebrain organoid, forming a vascular network within the brain organoid.

FIG. 3F depicts an embodiment of fluorescent images showing the development of CD31-positive brain capillaries within the forebrain organoid, and close association of GFAP-positive neural cells with the brain capillaries.

FIGS. 3G-3H depict an embodiment of fluorescent images showing the co-localization of claudin-5 and glucose transporter 1 (Glut-1) with the endothelial cells, which is indicative of an intact blood-brain barrier.

FIG. 3I depicts an embodiment of electron microscopy images of fused vascularized forebrain organoid cross-sections showing the presence of a brain capillary lumen, microvesicles (MV), tight junctions (TJ), and adherens junctions (AJ).

FIG. 3J depicts an embodiment of fluorescent images of Day 21 vascularized brain organoids immunostained for GFP, CD31, and Collagen IV. The fluorescent images show that the brain endothelium (CD31) was covered by a continuous basement membrane, an important structure for regulating angiogenesis and maintaining the BBB, which was defined by the molecular marker Collagen IV.

FIGS. 3K-3L depict an embodiment of fluorescent images showing the presence of GFAP-positive astrocytes in close association with GFP-expressing endothelial cells in the fused vascularized forebrain organoid.

FIG. 3M depicts an embodiment of fluorescent images showing the presence of AQP-4-postive astrocytes in the vascularized brain organoid, which are also importantly involved in blood-brain barrier function.

FIG. 3N-3O depicts an embodiment of fluorescent images showing the presence of newly formed capillaries ensheathed by astrocytic processes and by human pericytes (stained for PDGFR-β, FIG. 3N) in the vascularized brain organoid, indicative of the resemblance of in vivo human BBB-like structure with endothelial cells of the capillary wall connected through tight junctions, astrocytic end-feet and pericytes ensheathing. Also depicted in FIG. 3O is the co-localization of cadherin 5 (CDH5) with the endothelial cells, which is indicative of tight junctions of the blood-brain barrier.

FIG. 4A depicts an embodiment of single cell RNA sequencing of fused vascularized forebrain organoids. The data shows the presence of multiple cell types that make up the cerebrovascular network, including neurons, astrocytes, and endothelial cells.

FIG. 4B depicts an embodiment of the single cell RNA sequencing data showing the presence of multiple sub-clusters of endothelial cells, suggesting the presence of a diverse population of cells in the fused vascularized forebrain organoids.

FIG. 4C depicts an embodiment of a heat map showing the relative expression of cell markers in the cell types identified in the single cell RNA sequencing of the fused vascularized forebrain organoids. NPs1: neural progenitors; Ast: astrocytes; PAst: proliferative astrocytes, GABAs: GABAergic neurons; GluNs: glutaminergic neurons: VLMCs: vascular leptomeningeal cells; Brain VEs: brain vascular endothelial cells; VEs2: vascular endothelial cells 2; PAs: perivascular adipocytes; TCs: tendon cells; PCs: proliferative cells.

FIG. 4D depicts an embodiment of a violin plot showing the expression of various neuronal and vascular markers in the cell types identified in the single cell RNA sequencing of the fused vascularized forebrain organoids.

FIG. 4E depicts an embodiment of a map showing relative prevalence of potential interactions between protein receptors and ligands expressed by two different cell types identified in the single cell RNA sequencing of the fused vascularized forebrain organoids. The data points shown here relate to crosstalk from vascular cell types to vascular cell types. NPs1: neural progenitors 1; NPs2: neural progenitors 2; PPs: proliferative progenitors; GABAs: GABAergic neurons; GluNs: glutaminergic neurons; VLMCs: vascular leptomeningeal cells; VEs1: vascular endothelial cells 1; VEs2: vascular endothelial cells 2; Pas: perivascular adipocytes; TCs: tendon cells; PCs: proliferative cells.

FIG. 4F depicts an embodiment of a map showing relative prevalence of potential interactions between protein receptors and ligands expressed by two different cell types identified in the single cell RNA sequencing of the fused vascularized forebrain organoids. The data points shown here relate to crosstalk from neural cell types to vascular cell types.

FIG. 4G depicts an embodiment of a map showing relative prevalence of potential interactions between protein receptors and ligands expressed by two different cell types identified in the single cell RNA sequencing of the fused vascularized forebrain organoids. The data points shown here relate to crosstalk from vascular cell types to neural cell types.

FIG. 5A depicts an embodiment of single cell RNA sequencing data from Day 30 vascularized brain organoids. The data shows the presence of multiple cell types that make up a complete neurovascular unit, including excitatory neurons, inhibitory neurons, neural progenitors (NPs), astrocytes, endothelial cells (ECs), pericytes, mesenchymal stem cells (MSCs), smooth muscle cells (SMCs), and fibroblasts.

FIG. 5B depicts an embodiment of a gene expression analysis of endothelial cells in vascularized brain organoids. Gene expression data from endothelial cells in vascularized brain organoids was compared with gene expression data from organ-specific endothelial cells generated by the Tabula Muris Consortium. The data shows that endothelial cells in vascularized brain organoids presented an identical gene expression pattern with brain microvascular endothelial cells (BMECs) but not with other organ-specific endothelial cells, indicative of the endothelial acquisition of brain-specific transcriptomic signatures in vascularized brain organoids.

### DETAILED DESCRIPTION

The blood-brain barrier (BBB) offers a significant boundary to limit the exposure of the central nervous system from the rest of the body by regulating transport of essential molecules such as oxygen, carbon dioxide, and nutrients, but preventing the crossing of other molecules and larger biological entities such as cells and pathogens. The BBB is mediated by the formation of tight junctions between endothelial cells that make up blood vessels and capillaries in the brain. Additional cells in proximity such as astrocytes and pericytes also support the endothelial cells to maintain the BBB. For a model of the BBB to be representative of the natural structure, this highly organized structure forming a boundary between vascular and neuronal portions of the model is necessary.

Described herein are human blood-brain barrier models produced by vascularizing human brain organoids. These blood-brain barrier models may be used to model and study brain vascular disorders. These vascularized brain organoids may be transplanted in vivo, such as in the cortex of a mouse, to reconstitute active brain perfusion and integrate the organoid with living animals for advanced functional and in vivo study of the blood-brain barrier. These organoids also serve as a powerful drug screening platform to evaluate drug delivery across the blood-brain barrier.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as scrum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminocthane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx 15, ERas, ECAT15-1, ECAT15-2, Tcl1, β-Catenin, ECATI, Esg1, Dnmt3L, ECAT8, Gdf3, Fth117, Sal14, Rex1, UTFI, Stella, Stat3, Grb2, Prdm14, Nr5a1, Nr5a2, or E-cadherin, or any combination thereof. Other methods of producing induced pluripotent stem cells as conventionally known in the art are also envisioned.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), epiblast stem cells (EpiSC), and induced pluripotent stem cells.

In developmental biology, cellular differentiation is the process by which a less specialized cell becomes a more specialized cell type. As used herein, the term “differentiation” or “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of target stem cells.

### Cell Differentiation

In some embodiments, known methods for producing downstream cell types from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells or germ cells can originate from a variety of animal species including, but not limited to, various mammalian species including humans.

In some embodiments, the pluripotent stem cells are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the pluripotent stem cells are cultured in growth media that supports the growth of stem cells. In some embodiments, the pluripotent stem cells are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, iPSCs are expanded in cell culture. In some embodiments, iPSCs are expanded in Matrigel. In some embodiments, the iPSCs are expanded in cell culture comprising a ROCK inhibitor (e.g. Y-27632).

In some embodiments, pluripotent stem cells, mesoderm, vascular lineage cells, ectoderm, neural lineage cells, or any combination thereof, are contacted with a Wnt pathway activator or Wnt pathway inhibitor. In some embodiments, the Wnt pathway activator comprises a Wnt protein. In some embodiments, the Wnt protein comprises a recombinant Wnt protein. In some embodiments, the Wnt pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt pathway activator comprises a GSK3 pathway inhibitor. In some embodiments, the Wnt pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway inhibitor comprises IWR-1, C59, PNU 74654, KY-02111, PRI-724, FH-535, DIF-1, or XAV939, or any combination thereof. In some embodiments, the cells are not treated with a Wnt pathway activator or Wnt pathway inhibitor. The Wnt pathway activator or Wnt pathway inhibitor provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with an FGF pathway activator. In some embodiments, the FGF pathway activator comprises an FGF protein. In some embodiments, the FGF protein comprises a recombinant FGF protein. In some embodiments, the FGF pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF pathway activator. The FGF pathway activator provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with a BMP pathway activator or BMP pathway inhibitor. In some embodiments, the BMP pathway activator comprises a BMP protein. In some embodiments, the BMP protein is a recombinant BMP protein. In some embodiments, the BMP pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway inhibitor comprises Noggin, Dorsomorphin, RepSox, LY364947, LDN-193189, SB-431542, or any combination thereof. In some embodiments, the cells are not treated with a BMP pathway activator or BMP pathway inhibitor. The BMP pathway activator or BMP pathway inhibitor provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with a VEGF pathway activator. In some embodiments, the VEGF pathway activator comprises one or more of VEGF or GS4012. In some embodiments, the cells are not treated with a VEGF pathway activator. The VEGF pathway activator provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with a TGF-beta (TGF-b) pathway activator or TGF-b pathway inhibitor. In some embodiments, the TGF-b family comprises bone morphogenetic protein (BMP), growth and differentiation factor (GDF), anti-Müllerian hormone, Activin, and Nodal pathways. In some embodiments, the TGF-b pathway activator comprises TGF-b 1, TGF-b 2, TGF-b 3, Activin A, Activin B, Nodal, a BMP, IDE1, IDE2, or any combination thereof. In some embodiments, the TGF-b pathway inhibitor comprises A8301, RepSox, LY365947, SB-431542, or any combination thereof. In some embodiments, the cells are not treated with a TGF-b pathway activator or TGF-b pathway inhibitor. The TGF-b pathway activator or TGF-b pathway inhibitor provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with a cAMP pathway activator. In some embodiments, the cAMP pathway activator comprises forskolin or cAMP. In some embodiments, the cells are not treated with a cAMP pathway activator. The CAMP pathway activator provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with ascorbic acid. In some embodiments, the cells are not treated with ascorbic acid. Ascorbic acid as provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with leukemia inhibitory factor (LIF). In some embodiments, the cells are not treated with LIF. LIF as provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with glial cell line-derived neurotrophic factor (GDNF). In some embodiments, the cells are not treated with GDNF. GDNF as provided herein may be used in combination with any of the other growth factors, pathway activators, or pathway inhibitors provided herein.

In some embodiments, pluripotent stem cells, mesoderm, vascular lineage cells, ectoderm, neural lineage cells, or any combination thereof, are contacted with brain-derived neurotrophic factor (BDNF). In some embodiments, the cells are not treated with BDNF. BDNF as provided herein may be used in combination with any of the other growth factors, pathway activators, or pathway inhibitors provided herein.

In some embodiments, for any of the small molecule compounds, pathway activators, pathway inhibitors, or growth factors, the cells are contacted for a time that is, is about, is at least, is at least about, is not more than, or is not more than about, 1 hour, 2 hours, 3 hours, 4 hours, 5 hours, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 1 hour to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the PSCs are differentiated into mesoderm cells. In some embodiments, the PSCs are differentiated to vascular lineage cells. In some embodiments, the PSCs are differentiated to blood vessel organoids. In some embodiments, the PSCs are differentiated into ectoderm cells. In some embodiments, the PSCs are differentiated to neural lineage cells. In some embodiments, the PSCs are differentiated to cortical organoids.

In some embodiments, any of the cells disclosed herein may be cryopreserved for later use. In some embodiments, the cells are cryopreserved according to methods generally known in the art.

### Methods of Making Blood Vessel Organoid

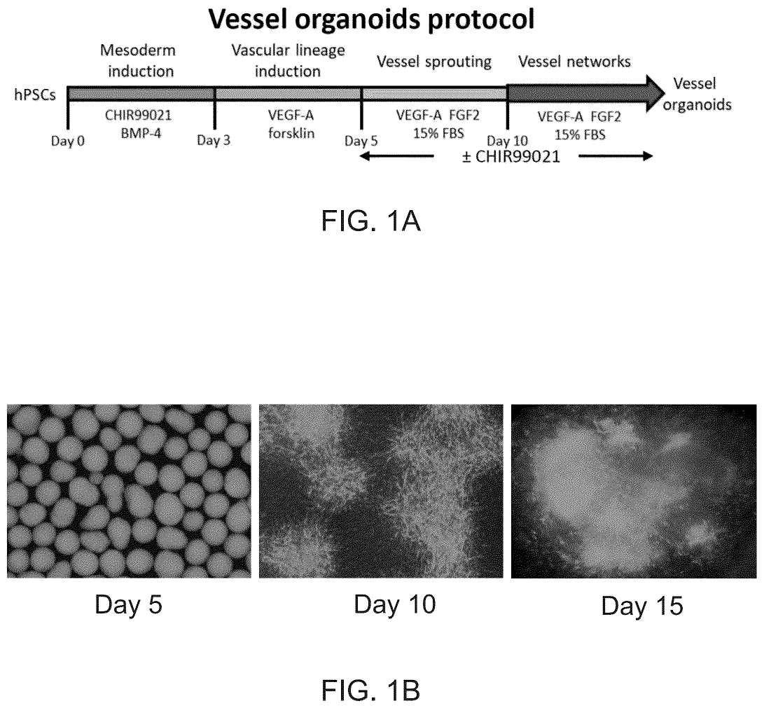

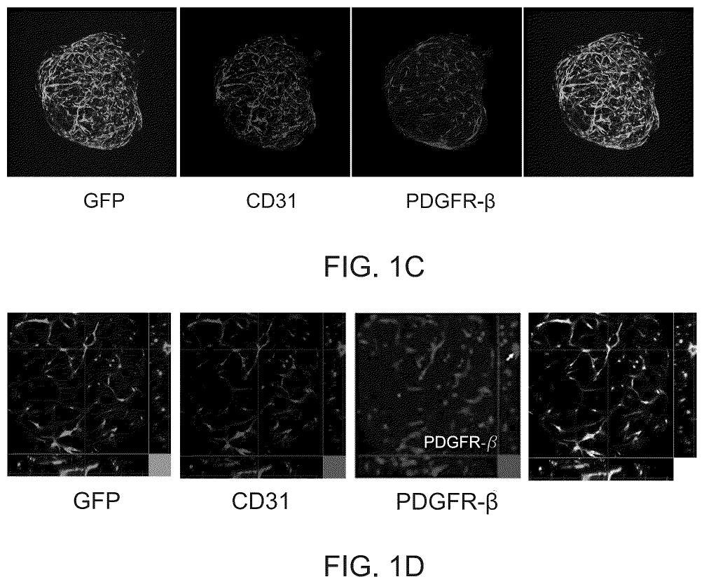

Exemplary methods for producing blood vessel (vascular) organoids from pluripotent stem cells may be found in Wimmer et al. Generation of blood vessel organoids from human pluripotent stem cells. Nature Protocols (2019) 14 (11): 3082-3100 and Wimmer et al. Human blood vessel organoids as a model of diabetic vasculopathy. Nature (2019) 565 (7740): 505-510, each of which is hereby expressly incorporated by reference in its entirety. A schematic for an improved method for producing blood vessel organoids from pluripotent stem cells is depicted in FIG. 1A. The methods may involve the use of a Wnt pathway activator such as CHIR99021 during differentiation to produce endothelial cells that resemble those that are found in brain blood vessels.

Disclosed herein are methods of producing blood vessel organoids. In some embodiments, the methods comprise contacting an angiogenic sprout with a Wnt pathway activator, an FGF pathway activator, a VEGF pathway activator, and optionally a growth serum, for a first period of time; thereby forming the blood vessel organoid. In some embodiments, the methods comprise contacting an angiogenic sprout with an FGF pathway activator, a VEGF pathway activator, optionally a Wnt pathway activator, and optionally a growth scrum, for a first period of time; thereby forming the blood vessel organoid.

In some embodiments, the angiogenic sprout is derived from pluripotent stem cells, for example, induced pluripotent stem cells. In some embodiments, the angiogenic sprout is cultured in a basement membrane matrix. In some embodiments, the angiogenic sprout is cultured in collagen I and/or Matrigel. In some embodiments, the first period of time is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any number of days within a range defined by any two of the aforementioned number of days, for example, 1-30 days, 1-10 days, 5-20 days, 10-30 days, or 5-25 days. In some embodiments, the first period of time is 5 days or at least 5 days. In some embodiments, the angiogenic sprout has been produced according to a method comprising: a) contacting pluripotent stem cells with a Wnt pathway activator and a BMP pathway activator for a second period of time to form vascular lineage cells; and b) contacting the vascular lineage cells with a VEGF pathway activator and a second cAMP pathway activator for a third period of time; thereby forming the angiogenic sprout. In some embodiments, the vascular lineage cells are cultured in a basement membrane matrix. In some embodiments, the vascular lineage cells are cultured in collagen I and/or Matrigel. In some embodiments, the second period of time is, is about, is at least, is at least about, is not more than, or is not more than about, 1, 2, 3, 4, or 5 days, or any number of days within a range defined by any two of the aforementioned number of days, for example, 1-5 days, 1-3 days, or 3-5 days. In some embodiments, the second period of time is 3 days. In some embodiments, the third period of time is, is about, is at least, is at least about, is not more than, or is not more than about, 1, 2, 3, or 4 days, or any number of days within a range defined by any two of the aforementioned number of days, for example, 1-4 days, 1-2 days, or 2-4 days. In some embodiments, the third period of time is 2 days.

In some embodiments, the BMP pathway activator is provided at a concentration that is, is about, is at least, is at least about, is not more than, or is not more than about, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, for example, 10-100 ng/mL, 10-30 ng/ml, 30-100 ng/ml, or 20-70 ng/mL. In some embodiments, the BMP pathway activator is provided at a concentration of 30 ng/ml or about 30 ng/ml. In some embodiments, the BMP pathway activator is BMP4.

In some embodiments, the Wnt pathway activator is provided at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1-20 μM, 1-12 μM, 4-12 μM, 4-20 μM, 2-6 μM, or 10-15 μM. In some embodiments, the Wnt pathway activator is provided at a concentration of 4 μM or about 4 μM. In some embodiments, the Wnt pathway activator is provided at a concentration of 12 μM or about 12 μM. In some embodiments, the Wnt pathway activator is CHIR99201.

In some embodiments, the second cAMP pathway activator is provided at a concentration that is, is about, is at least, is at least about, is not more than, or is not more than about, 0.5, 1, 1.5, 2, 2.5, 3, 3.5, or 4 μM, or any concentration within a range defined by any two of the aforementioned concentrations, for example, 0.5-4 μM, 0.5-2 μM, 2-4 μM or 1-3 μM. In some embodiments, the second cAMP pathway activator is provided at a concentration of 2 μM or about 2 μM. In some embodiments, the second cAMP pathway activator is forskolin.

In some embodiments, the growth serum is provided at a concentration that is, is about, is at least, is at least about, is not more than, or is not more than about, 0.5%, 1%, 2%, 3%, 4%, 5%, 6%, 7%, 8%, 9%, 10%, 11%, 12%, 13%, 14%, 15%, 16%, 17%, 18%, 19%, or 20%, or any concentration within a range defined by any two of the aforementioned concentrations, for example, 0.5%-20%, 0.5%-5%, 1%-15%, 10-15%, 15-20%, or 12-18%, In some embodiments, the growth serum is provided at 15% or about 15%. In some embodiments, the growth serum is provided at 1% or about 1%. In some embodiments, the growth serum is fetal bovine serum (FBS).

In some embodiments, the VEGF pathway activator is provided at a concentration that is, is about, is at least, is at least about, is not more than, or is not more than about, 10, 20, 30, 40, 50, 60, 70, 80, 90, 100, 110, 120, 130, 140, or 150 ng/mL, or any concentration within a range defined by any two of the aforementioned concentrations, for example 10-150 ng/mL, 10-100 ng/ml, 100-150 ng/mL, or 80-120 ng/mL. In some embodiments, the VEGF is provided at 100 ng/ml or about 100 ng/mL. In some embodiments, the VEGF pathway activator is VEGF.

In some embodiments, the FGF pathway activator is provided at a concentration that is, is about, is at least, is at least about, is not more than, or is not more than about, 10, 20, 30, 40, 50, 60, 70, 80, 90, 100, 110, 120, 130, 140, or 150 ng/mL, or any concentration within a range defined by any two of the aforementioned concentrations, for example 10-150 ng/ml, 10-100 ng/mL, 100-150 ng/ml, or 80-120 ng/mL. In some embodiments, the FGF pathway activator is provided at 100 ng/ml or about 100 ng/mL. In some embodiments, the FGF pathway activator is FGF2.

In some embodiments, the blood vessel organoid differs from a blood vessel organoid that has been produced without contacting the cells of step b) with the Wnt pathway activator in step c) by having increased expression of blood-brain barrier-specific endothelial markers. In some embodiments, the blood-brain barrier-specific endothelial markers comprise glucose transporter 1 (GLUT-1) and zonula occludens-1 (tight junction protein-1; ZO-1). In some embodiments, the blood vessel organoid comprises endothelial cells that express CD31 and pericyte cells, or progenitors thereof, that express PDGFR-β.

### Methods of Making Brain Organoid

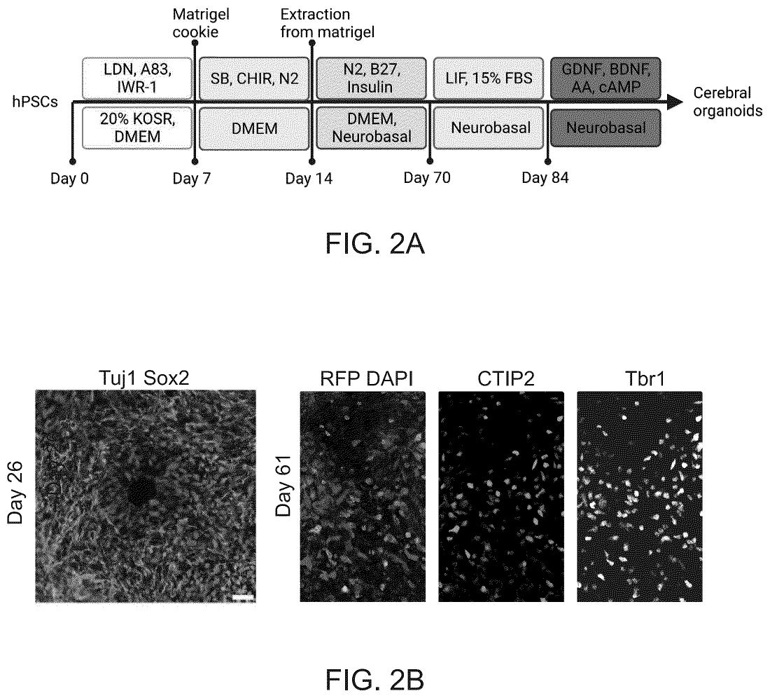

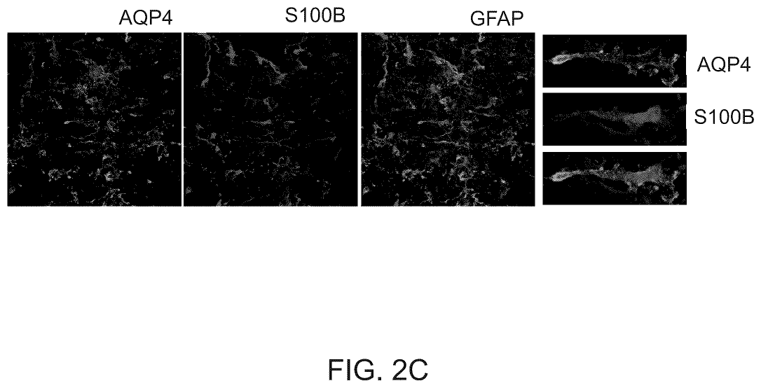

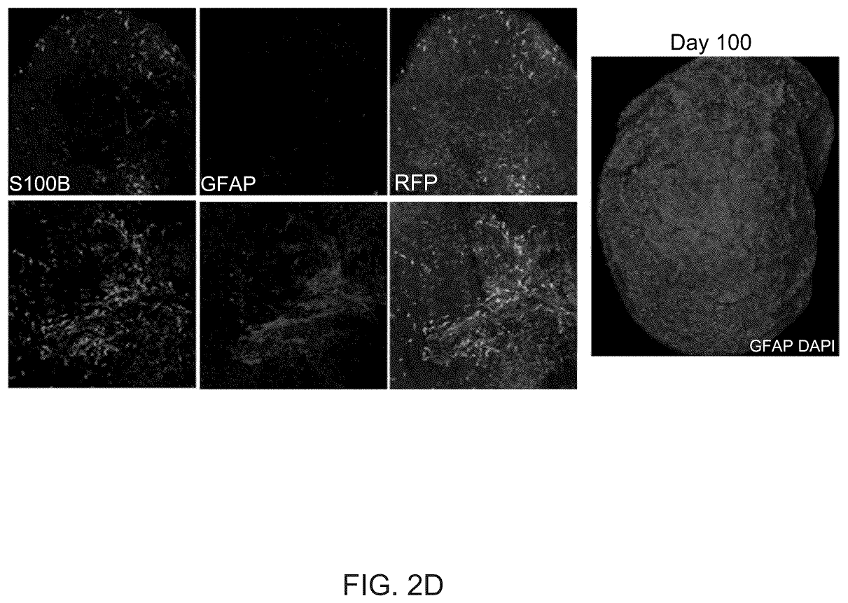

Exemplary methods for producing brain organoids from pluripotent stem cells may be found in Qian et al. Brain-Region-Specific Organoids Using Mini-bioreactors for Modeling ZIKV Exposure. Cell (2016) 165 (5): 1238-1254 and Qian et al. Generation of human brain region-specific organoids using a miniaturized spinning bioreactor. Nature Protocols (2018) 13 (3): 565-580, each of which is expressly incorporated by reference in its entirety. A schematic for an exemplary method for producing dorsal forebrain organoids, which are a type of cortical (brain) organoid, is provided in FIG. 2A. These methods can be adapted to produce cortical organoids of alternative types, such as midbrain, striatal brain, hypothalamus, hippocampal, or spinal cord organoids. The methods may involve the use of LIF and/or fetal bovine serum during differentiation to induce formation of astrocytes in the brain organoids.

Disclosed herein are methods of producing brain organoids. In some embodiments, the methods comprise a) contacting pluripotent stem cells with a BMP pathway inhibitor, a TGF-beta pathway inhibitor, and a Wnt pathway inhibitor for a first period of time to form neuroectoderm cells; b) contacting the neuroectoderm cells of step a) with a second TGF-beta pathway inhibitor, and a Wnt pathway activator for a second period of time to form neuroepithelium cells; c) contacting the neuroepithelium cells of step b) with insulin for a third period of time to form cerebral tissue organoids; and d) contacting the cerebral tissue organoids of step c) with GDNF, BDNF, ascorbic acid, and a cAMP pathway activator for a fourth period of time to form the brain organoid. In some embodiments, the cerebral tissue organoid is further contacted with LIF and growth serum for a portion of the fourth period of time to induce astrocyte proliferation in the brain organoid. In some embodiments, the first period of time is, is about, is at least, is at least about, is not more than, or is not more than about, 1, 2, 3, 4, 5, 6, 7, 8, 9, 10, 11, 12, 13, or 14 days, or any number of days within a range defined by any two of the aforementioned number of days, for example, 1-14 days, 1-7 days, 7-14 days, or 5-10 days. In some embodiments, the first period of time is 7 days. In some embodiments, the second period of time is, is about, is at least, is at least about, is not more than, or is not more than about, 1, 2, 3, 4, 5, 6, 7, 8, 9, 10, 11, 12, 13, or 14 days, or any number of days within a range defined by any two of the aforementioned number of days, for example, 1-14 days, 1-7 days, 7-14 days, or 5-10 days. In some embodiments, the second period of time is 7 days. In some embodiments, the third period of time is, is about, is at least, is at least about, is not more than, or is not more than about, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days, for example, 20-70 days, 20-60 days, 40-70 days, or 40-60 days. In some embodiments, the third period of time is 56 days. In some embodiments, the fourth period of time is, is about, is at least, is at least about, is not more than, or is not more than about, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days, for example, 7-70 days, 7-50 days, 20-60 days, or 20-70 days. In some embodiments, the portion of the fourth period of time is, is about, is at least, is at least about, is not more than, or is not more than about, 7, 8, 9, 10, 11, 12, 13, 14, 15, 16, 17, 17, 18, 19, 20, or 21 days, or any number of days within a range defined by any two of the aforementioned number of days, for example, 7-21 days, 7-14 days, 14-21 days, or 10-18 days. In some embodiments, the portion of the fourth period of time is 14 days. In some embodiments, the portion of the fourth period of time is at the beginning of the fourth period of time. In some embodiments, the brain organoid comprises cells that express Tuj1, Sox2, Ctip1, Tbr1, or any combination thereof. In some embodiments, the brain organoid comprises astrocytes that express S100B, GFAP, and AQP4.

In some embodiments, the BMP pathway inhibitor is provided at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, for example, 0.1-2 μM, 0.1-1 μM, 1-2 μM, or 0.5-1.5 μM. In some embodiments, the BMP pathway inhibitor is provided at a concentration of 1 μM or about 1 μM. In some embodiments, the BMP pathway inhibitor is LDN-193189.

In some embodiments, the TGF-beta pathway inhibitor and the second TGF-beta pathway inhibitor is the same or different. In some embodiments, the TGF-beta pathway inhibitor is provided at a concentration that is, is about, is at least, is at least about, is not more than, or is not more than about, 0.5, 1, 1.5, 2, 2.5, 3, 3.5, or 4 μM, or any concentration within a range defined by any two of the aforementioned concentrations, for example, 0.5-4 μM, 0.5-2 μM, 2-4 μM, or 1-3 μM. In some embodiments, the TGF-beta pathway inhibitor is provided at a concentration of 2 μM or about 2 μM. In some embodiments, the second TGF-beta pathway inhibitor is provided at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, for example, 0.1-2 μM, 0.1-1 μM, 1-2 μM, or 0.5-1.5 μM. In some embodiments, the second TGF-beta pathway inhibitor is provided at a concentration of 1 μM or about 1 μM. In some embodiments, the TGF-beta pathway inhibitor and/or the second TGF-beta pathway inhibitor is A83-01. In some embodiments, the TGF-beta pathway inhibitor and/or the second TGF-beta pathway inhibitor is SB-431542.

In some embodiments, the Wnt pathway inhibitor is provided at a concentration that is, is about, is at least, is at least about, is not more than, or is not more than about, 0.5, 1, 1.5, 2, 2.5, 3, 3.5, 4, 4.5, or 5 μM, or any concentration within a range defined by any two of the aforementioned concentrations, for example, 0.5-5 μM, 0.5-3 μM, 3-5 μM, or 2-4 μM. In some embodiments, the Wnt pathway inhibitor is provided at a concentration of 3 μM or about 3 μM. In some embodiments, the Wnt pathway inhibitor is IWR-1.

In some embodiments, the Wnt pathway activator is provided at a concentration that is, is about, is at least, is at least about, is not more than, or is not more than about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, for example, 0.1-2 μM, 0.1-1 μM, 1-2 μM, or 0.5-1.5 μM. In some embodiments, the Wnt pathway activator is provided at a concentration of 1 μM or about 1 μM. In some embodiments, the Wnt pathway activator is CHIR99021.

In some embodiments, the insulin is provided at a concentration that is, is about, is at least, is at least about, is not more than, or is not more than about, 0.5, 1, 1.5, 2, 2.5, 3, 3.5, 4, 4.5, or 5 μg/mL, or any concentration within a range defined by any two of the aforementioned concentrations, for example, 0.5-5 ug/mL, 0.5-2.5 ug/mL, 2.5-5 ug/mL, or 1-3 μg/mL. In some embodiments, the insulin is provided at a concentration of 2.5 μg/mL or about 2 μg/mL.

In some embodiments, the LIF is provided at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 mg/mL, for example, 1-20 mg/mL, 1-10 mg/mL, 10-20 mg/mL, or 5-15 mg/mL. In some embodiments, the LIF is provided at a concentration of 10 mg/mL or about 10 mg/mL.

### Vascularized Brain Organoids and Methods of Making Vascularized Brain Organoids

Disclosed herein are methods for producing a vascularized brain organoid. In some embodiments, the methods comprise contacting a blood vessel organoid and a brain organoid; and culturing the blood vessel organoid and the brain organoid for a period of time until the blood vessel organoid and the brain organoid fuse together and blood vessels of the blood vessel organoid infiltrate the brain organoid, where neurons of the brain organoid innervate the blood vessels of the blood vessel organoid that have infiltrated the organoid, thereby forming the vascularized brain organoid. In some embodiments, the vascularized brain organoid comprises a blood-brain barrier that is formed between the brain organoid and all or a portion of the blood vessels of the blood vessel organoid that have infiltrated the brain organoid. In some embodiments, the blood-brain barrier comprises endothelial cells linked with tight junctions, astrocytes, and pericytes. In some embodiments, the endothelial cells express CD31, GLUT-1 and PDGFR-β. In some embodiments, the tight junctions comprise claudin-5. In some embodiments, the endothelial cells express CD31, GLUT-1 and PDGFR-β. In some embodiments, the tight junctions comprise claudin-5, ZO-1 and cadherin 5. In some embodiments, the astrocytes express S100B, GFAP, and AQP4. In some embodiments, the pericytes express PDGFR-β, a-smooth muscle actin (αSMA), and neural/glial antigen 2 (NG2). In some embodiments, the endothelial cells form a continuous basement membrane and express collagen IV. In some embodiments, the vascularized brain organoid comprises cells selected from the group consisting of neural progenitors, proliferative astrocytes, GABAergic neurons, glutamatergic neurons, proliferative cells, brain vascular endothelial cells, vascular leptomeningeal cells, perivascular adipocytes, and tendon cells. In some embodiments, the vascularized brain organoid comprises cells selected from the group consisting of neural progenitor cells, GABAergic neurons, glutamatergic neurons, proliferative astrocytes, proliferative GABAergic neurons, mesenchymal stem cells, endothelial cells, pericytes, vascular smooth muscle cells, fibroblast, and proliferative cells. In some embodiments, the cells of the vascular brain organoid are identified by cell-type specific gene expression markers. In some embodiments, the cells of the vascular brain organoid are identified by cell-type specific gene expression markers. In some embodiments, the blood vessels comprise capillaries. In some embodiments, the capillaries are ensheathed by pericytes and end-feet of the astrocytes. In some embodiments, the brain organoid is a forebrain organoid, a midbrain organoid, a hypothalamus organoid, a hippocampus organoid, a spinal cord organoid, or a striatal brain organoid. In some embodiments, the blood vessel organoid and the brain organoid are contacted and/or cultured in a basement membrane matrix or component thereof, optionally Matrigel. In some embodiments, the blood vessel organoid and the brain organoid are cultured for a period of time that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days, for example, 1-70 days, 1-50 days, 30-70 days, or 30-60 days. In some embodiments, the blood vessel organoid and the brain organoid are cultured with agitation for at least a portion of the period of time. In some embodiments, the agitation comprises shaking. In some embodiments, the blood vessel organoid and the brain organoid are cultured: 1) without agitation for 1, 2, 3, 4, 5, 6, 7, 8, 9, 10, 11, 12, 13, or 14 days, or any number of days within a range defined by any two of the aforementioned days, for example, 1-14 days, 1-7 days, 7-14 days, or 5-10 days; and subsequently 2) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days, for example, 1-70 days, 1-50 days, 30-70 days, or 30-60 days. In some embodiments, the blood vessel organoid and the brain organoid are cultured in a medium that promotes neuronal growth and/or vascular growth. In some embodiments, the blood vessel organoid and the brain organoid are cultured in a medium that comprises growth factors that promote neuronal growth and/or growth factors that promote vascular growth. In some embodiments, the growth factors that promote neuronal growth comprise a cAMP pathway activator, ascorbic acid, BDNF, GDNF, or any combination thereof. In some embodiments, the growth factors that promote vascular growth comprise growth serum, a VEGF pathway activator, an FGF pathway activator, or any combination thereof. In some embodiments, the blood vessel organoid has been produced according to methods provided herein or adaptations of methods generally known in the art. In some embodiments, the brain organoid has been produced according to methods provided herein or adaptations of methods generally known in the art.

In some embodiments, culturing the blood vessel organoid and the brain organoid comprises: a) culturing the blood vessel organoid and the brain organoid without agitation for 4, 5, 6, 7, 8, 9, or 10 days; and b) culturing the organoids of step a) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. In some embodiments, the organoids of step a) and step b) are cultured in a medium comprising growth factors that promote neuronal growth and/or growth factors that promote vascular growth. In some embodiments, the blood vessel organoid and the brain organoid are cultured without agitation in step a) for 7 days. In some embodiments, the organoids of step a) are cultured with agitation in step b) for at least 30 days. In some embodiments, the agitation comprises shaking. In some embodiments, the growth factors that promote neuronal growth comprise a cAMP pathway activator, ascorbic acid, BDNF, GDNF, or any combination thereof. In some embodiments, the growth factors that promote vascular growth comprise FBS, VEGF, FGF2, or any combination thereof. In some embodiments, the blood vessel organoid and the brain organoid are cultured in a basement membrane matrix or component thereof, for example, Matrigel.

In some embodiments of any of the methods disclosed herein, the cAMP pathway activator is provided at a concentration that is, is about, is at least, is at least about, is not more than, or is not more than about, 10, 20, 30, 40, 50, 60, 70, 80, 90, 100, 110, 120, 130, 140, or 150 μM, or any concentration within a range defined by any two of the aforementioned concentrations, for example, 10-150 μM, 10-50 μM, 50-150 μM, or 20-100 μM. In some embodiments, the cAMP pathway activator is provided at 50 μM or about 50 μM. In some embodiments, the cAMP pathway activator is cAMP.

In some embodiments of any of the methods disclosed herein, the ascorbic acid is provided at a concentration that is, is about, is at least, is at least about, is not more than, or is not more than about, 50, 60, 70, 80, 90, 100, 110, 120, 130, 140, 150, 160, 170, 180, 190, 200, 210, 220, 230, 240, 250, 260, 270, 280, 290, or 300 μM, or any concentration within a range defined by any two of the aforementioned concentrations, for example, 50-300 μM, 50-200 μM, 200-300 μM, or 150-250 μM. In some embodiments, the ascorbic acid is provided at 200 μM or about 200 μM.

In some embodiments of any of the methods disclosed herein, the BDNF is provided at a concentration that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, for example, 1-30 ng/ml, 10-20 ng/ml, 20-30 ng/ml, or 15-25 ng/mL. In some embodiments, the BDNF is provided at 20 ng/ml or about 20 ng/mL.

In some embodiments of any of the methods disclosed herein, the GDNF is provided at a concentration that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, for example, 1-30 ng/ml, 10-20 ng/ml, 20-30 ng/ml, or 15-25 ng/mL. In some embodiments, the GDNF is provided at 20 ng/ml or about 20 ng/mL.

In some embodiments of any of the methods disclosed herein, the growth serum is provided at a concentration that is, is about, is at least, is at least about, is not more than, or is not more than about, 0.5%, 1%, 2%, 3%, 4%, 5%, 6%, 7%, 8%, 9%, 10%, 11%, 12%, 13%, 14%, 15%, 16%, 17%, 18%, 19%, or 20%, or any concentration within a range defined by any two of the aforementioned concentrations, for example, 0.5%-20%, 0.5%-5%, 1%-15%, 10-15%, 15-20%, or 12-18%, In some embodiments, the growth serum is provided at 15% or about 15%. In some embodiments, the growth serum is provided at 1% or about 1%. In some embodiments, the growth serum is fetal bovine serum (FBS).

In some embodiments of any of the methods disclosed herein, the VEGF pathway activator is provided at a concentration that is, is about, is at least, is at least about, is not more than, or is not more than about, 10, 20, 30, 40, 50, 60, 70, 80, 90, 100, 110, 120, 130, 140, or 150 ng/mL, or any concentration within a range defined by any two of the aforementioned concentrations, for example 10-150 ng/ml, 10-100 ng/ml, 100-150 ng/ml, or 80-120 ng/ml. In some embodiments, the VEGF is provided at 100 ng/ml or about 100 ng/ml. In some embodiments, the VEGF pathway activator is VEGF.

In some embodiments of any of the methods disclosed herein, the FGF pathway activator is provided at a concentration that is, is about, is at least, is at least about, is not more than, or is not more than about, 10, 20, 30, 40, 50, 60, 70, 80, 90, 100, 110, 120, 130, 140, or 150 ng/ml, or any concentration within a range defined by any two of the aforementioned concentrations, for example 10-150 ng/ml, 10-100 ng/ml, 100-150 ng/mL, or 80-120 ng/ml. In some embodiments, the FGF pathway activator is provided at 100 ng/ml or about 100 ng/ml. In some embodiments, the FGF pathway activator is FGF2.

In some embodiments of any of the methods disclosed herein, the blood vessel organoid and/or the brain organoid are derived from pluripotent stem cells. In some embodiments, the pluripotent stem cells are embryonic stem cells or induced pluripotent stem cells. In some embodiments, the blood vessel organoid and/or the brain organoid are human. In some embodiments, the blood vessel organoid and/or the brain organoid have been derived from a subject, such as a human subject. In some embodiments, the subject comprises a cerebrovascular disease or a disease associated with blood-brain barrier dysfunction, for example, cerebral cavernous malformation, Alzheimer's disease, or amyotrophic lateral sclerosis.

Also disclosed herein are the vascularized brain organoids produced by any of the methods disclosed herein. In some embodiments, a vascularized brain organoid comprises endothelial cells linked with tight junctions, astrocytes, and pericytes. In some embodiments, the endothelial cells express CD31, GLUT-1 and PDGFR-β; the tight junctions comprise claudin-5, ZO-1 and cadherin 5; the astrocytes express S100B, GFAP, and AQP4; and the pericytes express PDGFR-β, αSMA and NG2. In some embodiments, the endothelial cells form a continuous basement membrane and express collagen IV. In some embodiments, the vascularized brain organoid comprises cells selected from the group consisting of neural progenitors, proliferative astrocytes, GABAergic neurons, glutamatergic neurons, proliferative cells, brain vascular endothelial cells, vascular leptomeningeal cells, perivascular adipocytes, and tendon cells. In some embodiments, the vascularized brain organoid comprises cells selected from the group consisting of neural progenitor cells, GABAergic neurons, glutamatergic neurons, proliferative astrocytes, proliferative GABAergic neurons, mesenchymal stem cells, endothelial cells, pericytes, vascular smooth muscle cells, fibroblast, and proliferative cells. In some embodiments, the cells are identified by cell-type specific gene expression markers. In some embodiments, the blood vessels comprise capillaries. In some embodiments, the capillaries are ensheathed by pericytes and end-feet of the astrocytes.

### Methods of Use

Also disclosed herein are methods of treating a cerebrovascular disease or a disease associated with blood-brain barrier dysfunction in a subject in need thereof. In some embodiments, the methods comprise administering any of the vascularized brain organoids disclosed herein, or a portion or fragment thereof, to the subject

Also disclosed herein are methods of screening. In some embodiments, the methods comprise contacting any of the vascularized brain organoids disclosed herein, or portions thereof, with a candidate compound or composition, and assessing the effects of the candidate compound or composition on the vascularized brain organoid or portions thereof. In some embodiments, the effect comprises transport of the candidate compound or composition across the blood-brain barrier of the organoid or portions thereof. In some embodiments, the vascularized brain organoid is a model for a cerebrovascular disease or a disease associated with blood-brain barrier dysfunction, and assessing the effects of the candidate compound or composition on the vascularized organoid comprises assessing the effects of the candidate compound or composition on the cerebrovascular disease or the disease associated with blood-brain barrier dysfunction. In some embodiments, the vascularized brain organoid has been produced from cells derived from a subject. In some embodiments, the cells derived from the subject are pluripotent stem cells. In some embodiments, the subject has or is disposed to develop the cerebrovascular disease or the disease associated with blood-brain barrier dysfunction.

### EXAMPLES

### Example 1. Generation of Blood Vessel (Vascular) Organoids

Exemplary methods for producing blood vessel (vascular) organoids from pluripotent stem cells may be found in Wimmer et al. Generation of blood vessel organoids from human pluripotent stem cells. Nature Protocols (2019) 14 (11): 3082-3100 and Wimmer et al. Human blood vessel organoids as a model of diabetic vasculopathy. Nature (2019) 565 (7740): 505-510, each of which is hereby expressly incorporated by reference in its entirety. A schematic for an improved method for producing blood vessel organoids from pluripotent stem cells is depicted in FIG. 1A.

Preparation of Feeder-Free iPSC Culture:

Matrigel was thawed on ice for up to 1 hour. 100 μL of thawed Matrigel was diluted in 6 mL of cold DMEM/F12 (for a 6 well plate). The Matrigel dilution was mixed well and used to coat a 6 well plate with 1 mL of Matrigel mixture per well. The plate was placed in a 37° C. incubator for up to 2 hours or overnight. iPSCs grown to 60-80% confluence were washed with approximately 2 mL of room temperature phosphate buffered saline (PBS). 1 mL of ReleSR dissociation reagent (StemCell Technologies) was added per well containing iPSCs and after 1 minute, most of the ReleSR was aspirated out, leaving some to keep iPSCs covered. The iPSCs were incubated in the ReleSR reagent for 6-8 minutes at room temperature. The sides of the iPSC plate were tapped to detach the iPSCs. 1 mL of mTeSR Plus medium (StemCell Technologies) was added per well to neutralize the ReleSR dissociation reagent. The plate was shaken gently to wash the iPSCs. The dissociated iPSCs were transferred to a 15 mL tube and dissociated to single cells by pipetting. The Matrigel mixture was aspirated from the coated 6 well plate and 2 mL of mTeSR Plus per well was added to the Matrigel plate. iPSCs were added to the Matrigel-coated plate at the desired density (1:20-1:50 dilution, or 10,000-30,000 cells per well). The plate was shaken briefly and returned to a 37° C. incubator. The growth medium was changed every day until the iPSCs reached approximately 80% confluence (5-7 days). These iPSCs can be used downstream for blood vessel organoid differentiation or passaged again for later use.

### Preparation of Blood Vessel Organoids:

One day prior to initiating the blood vessel organoid differentiation process (Day −1), iPSCs were seeded onto an Aggrewell 400 (StemCell Technologies) 24-well plate at 1.2×10⁶ cells per plate in aggregation medium (KnockOut DMEM/F12, 99 μM β-mercaptoethanol, Knockout Serum Replacement, 1× Glutamax, 1x non-essential amino acids (NEAA), 1x penicillin-streptomycin) supplemented with 50 μM Y-27632 (ROCK inhibitor), to form uniform stem cell aggregates.

On the first day (Day 0), the iPSCs were induced to differentiate into mesoderm by culturing the aggregates in N2B27 medium (50% DMEM/F12, 50% neurobasal medium, 99 μM β-mercaptoethanol, 1× Glutamax, 1x penicillin-streptomycin, 1x B27 supplement, 1x N2 supplement) supplemented with 12 μM CHIR99021 and 30 ng/ml BMP4.

After four days of mesoderm induction (Day 3), the differentiated mesoderm cells were induced to differentiate into a vascular lineage by culturing the mesoderm cells in N2B27 medium supplemented with 100 ng/mL VEGF and 2 μM forskolin.

After 2 days of vascular induction (Day 5), the differentiated vascular lineage cells were further cultured to develop formation of blood vessels.

For one 12 well plate, 5 mL of a 2 mg/mL collagen I solution was prepared by mixing 300 μL of 0.1 N NaOH, 450 μL of ddH₂O, 313 μL of 10×DMEM, 63 μL of HEPES, 49 μL of 7.5% sodium bicarbonate, 31 μL of Glutamax, 460 μL of Ham's F-12, and 3.33 mL of a 3 mg/mL collagen stock solution (PureCol; Advanced Biomatrix). The pH of this collagen I solution should be 7.4. Subsequently, a 4:1 collagen I solution to Matrigel mixture was prepared by mixing 4.5 mL of the collagen I solution with 1.5 mL of growth factor reduced Matrigel on ice.

0.5 mL of the collagen I/Matrigel mixture was used to coat the wells of a 12 well plate and incubated at 37° C. for 2 hours to solidify the mixture. The vascular lineage cells were resuspended in another fresh batch of collagen I/Matrigel mixture (unsolidified), and 0.5 mL of the cell suspension in collagen I/Matrigel was used to seed each well of the collagen I/Matrigel coated plates. The plate was returned to a 37° C. incubator for 2 hours to solidify the collagen I/Matrigel mixture containing vascular lineage cells. Subsequently, complete StemPro-34 serum free medium (Thermo Fisher) supplemented with 15% fetal bovine serum (FBS), 4 μM CHIR99021, 100 ng/mL VEGF, and 100 ng/mL FGF2 was added to the vascular lineage cells.

Over the course of 5 days of culturing in StemPro-34 media containing 15% FBS, 4 μM CHIR99021, 100 ng/mL VEGF, and 100 ng/ml FGF2, the vascular lineage cells mature to form blood vessel organoids comprising vascular networks. These blood vessel organoids may be used for optional downstream studies, such as isolating the blood vessels from the organoids, or transplant of the organoids in vivo.

In FIG. 1B, stem cells engineered to express GFP were differentiated to blood vessel organoids according to the method provided herein. The cells of the blood vessel organoids were organized in a vascular network. As shown in FIGS. 1C-1D, the cells of the blood vessel organoids expressed platelet endothelial cell adhesion molecule (PECAM-1; CD31) and platelet-derived growth factor receptor beta (PDGFR-β), which are markers for early endothelial cells. PDGFR-β is also expressed by pericyte progenitor cells, which give rise to pericytes that are involved in the blood-brain barrier. In FIG. 1D, endothelial tube structures with a lumen were present in the blood vessel organoids.

### Example 2. Generation of Cortical Organoids

Exemplary methods for producing blood vessel organoids from pluripotent stem cells may be found in Qian et al. Brain-Region-Specific Organoids Using Mini-bioreactors for Modeling ZIKV Exposure. Cell (2016) 165 (5): 1238-1254 and Qian et al. Generation of human brain region-specific organoids using a miniaturized spinning bioreactor. Nature Protocols (2018) 13 (3): 565-580, each of which is expressly incorporated by reference in its entirety. A schematic for an exemplary method for producing dorsal forebrain organoids, which are a type of cortical organoid, is provided in FIG. 2A. These methods can be adapted to produce cortical organoids of alternative types, such as midbrain, striatal brain, hypothalamus, hippocampal, and spinal cord organoids.

On the first day (Day 0), iPSCs were seeded on to Aggrewell 800 24-well plates (StemCell Technologies) at a final cell density of approximately 4×10⁶ cells per Aggrewell plate in 2 mL of Essential 8 medium (Thermo Fisher) supplemented with 10 μM Y-27632 (ROCK inhibitor). The Aggrewell was centrifuged at 100×g for 3 minutes at 4° C. to collect the stem cells at the bottom of the Aggrewell microwells. The Aggrewell may be first washed with Anti-Adherence Rinsing solution (StemCell Technologies) and washed with Essential 8 medium prior to adding the cells. The use of the Aggrewell plate is optional, but the use of this plate helps attain greater final numbers of organoids with more uniform size. A standard low attachment plate may also be used, which will result in larger organoids.

After 1 day of culture (Day 1) (or 2 days if the embryoid bodies are not large enough), the Aggrewell culture were agitated with a pipette to resuspend the embryoid bodies (EBs), and the resuspension was transferred to 15 mL tube. The EBs were washed with fresh DMEM/F12, resuspended in H1 medium (77% DMEM/F12, 20% KnockOut Serum Replacement medium, 1× Glutamax, 1× NEAA, 1× β-mercaptoethanol, 1× penicillin-streptomycin supplemented with 1 μM LDN-193189, 2 μM A83-01, and 3 μM IWR-1), and seeded onto a ultra-low attachment 6 well plate. The EBs were allowed to settle, the medium was aspirated away, and 3 mL of H1 medium supplemented with 10 μM Y-27632 was added to each well. The plate was incubated with shaking (120 rpm) at 37° C. for 48 hours. After the 48 hours, the medium was replaced with 3 mL of fresh H1 medium (without the ROCK inhibitor). After another 24 hours (Day 3 of culture), the medium was again changed with fresh HI medium (without the ROCK inhibitor).

After 4 days of culture in H1 medium (Day 4), the medium was changed out for H1 medium without IWR-1 and cultured for an additional 2 days with shaking (120 rpm) at 37° C.

After 2 days (Day 7), the resultant neuroectoderm cells were transferred to a 1.5 mL tube and allowed to settle. The supernatant was removed and the EBs were washed twice with 1 mL of F2 medium (DMEM/F12, 1x N2, 1x Glutamax, 1× NEAA, 1× β-mercaptocthanol, 1× penicillin-streptomycin supplemented with 1 μM SB-431542 and 1 μM CHIR99021). 67 μL of the resuspended EB in F12 medium (containing less than 60 EBs) were transferred to a fresh tube and combined with 100 μL of Matrigel. The EB/Matrigel mixture was added to a plate and solidified in a 37° C. incubator for 30 minutes. 3 mL of F2 medium was carefully added to the wells containing the EB/Matrigel droplets and returned to a 37° C. incubator without shaking for 48 hours. Every two days (Days 9, 11, 13) for a total of 6 days, the medium was replaced with fresh F2 medium.

At Day 14, the resultant neuroepithelium cells were released from the Matrigel and resuspended in 1-3 mL of H3 medium (50% DMEM/F12, 50% neurobasal medium, 1× N2, 1× B27, 1x Glutamax, 1× NEAA, 1× β-mercaptoethanol, 1× penicillin-streptomycin supplemented with 2.5 μg/mL insulin). The EB resuspension in H3 medium was added to a regular 6 well plate, and an additional 4.5 mL of H3 medium was added to each well. The cells were incubated with shaking (120 rpm) at 37° C.

Over the course of 54 days (Days 16-70), the medium is changed for fresh H3 medium every 2 days.

When the cerebral tissue organoids grow to a diameter of more than 1 mm (after Day 70), the medium is changed out to 3 mL F4 medium (neurobasal medium, 1× B27, 1x Glutamax, 1× NEAA, 1× 1x β-mercaptoethanol, 1× penicillin-streptomycin supplemented with 0.05 mM cAMP, 0.2 mM ascorbic acid, 20 ng/mL BDNF, and 20 ng/mL GDNF) to differentiate the cerebral tissue to cortical forebrain organoids. The medium is changed for fresh F4 medium every 2 days.

For astrocyte induction, the F4 medium used any time after Day 70 is supplemented with 1% FBS (which can be increased up to 15%) and 10 mg/mL leukemia inhibitory factor (LIF) for a span of 2 weeks.

As shown in FIG. 2B, the forebrain organoid produced according to the method herein expressed the neuronal cell marker class III beta-tubulin (Tuj1) and neural stem cell marker SRY-Box transcription factor (Sox2) as detected at Day 26 of culture, and the neural cell markers B-cell lymphoma/leukemia 11B (BCL11B; Ctip1) and T-Box brain transcription factor 1 (Tbr1) as detected at Day 61 of culture.

FIGS. 2C-2D shows images of a forebrain organoid further cultured with 1% FBS and 10 mg/mL LIF for 2 weeks to induce astrocyte formation. Astrocyte presence was confirmed by the detection of astrocyte markers S100 calcium binding protein B (S100B), glial fibrillary acidic protein (GFAP), and aquaporin 4 (AQP4).

### Example 3. Generation of Fused Vascularized Cortical Organoids

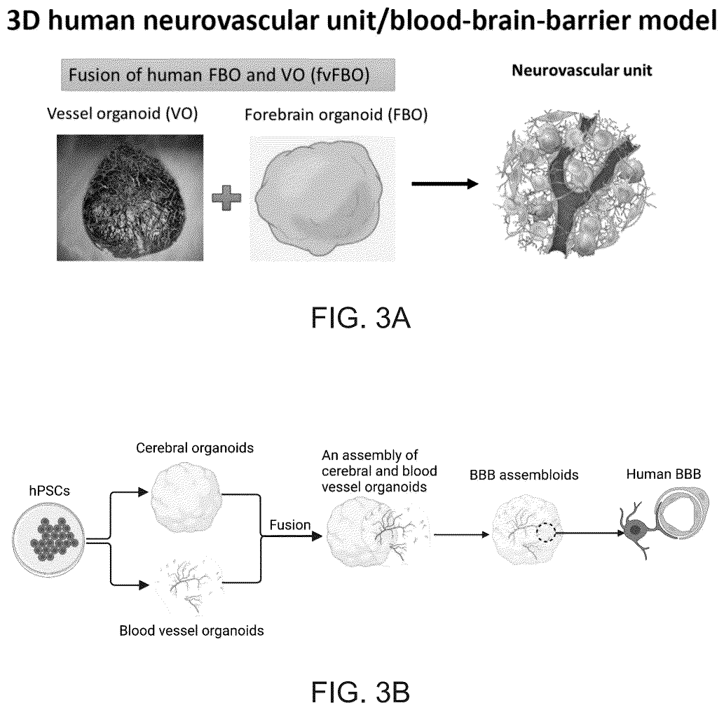

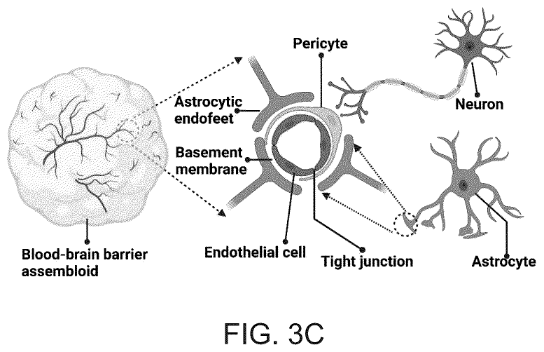

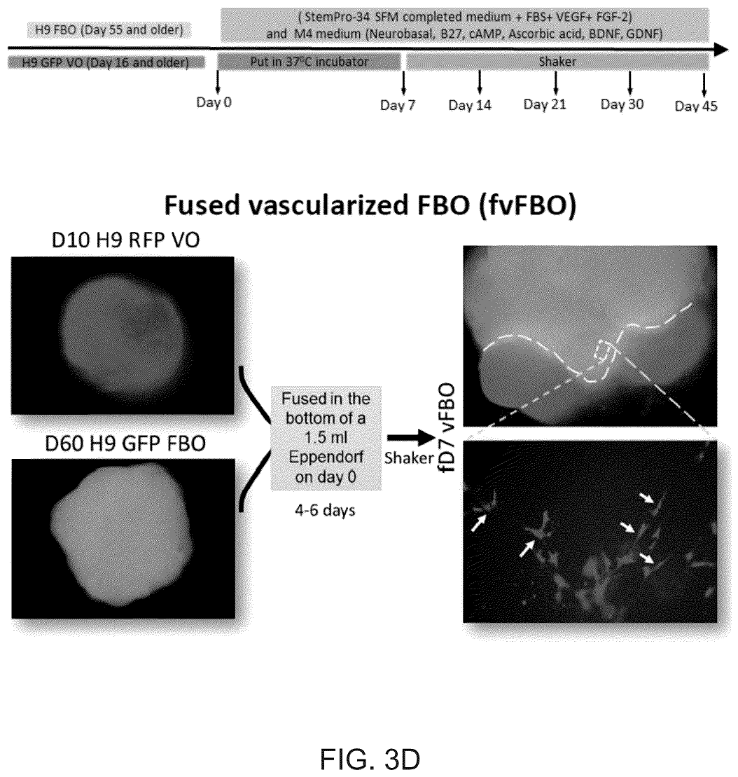

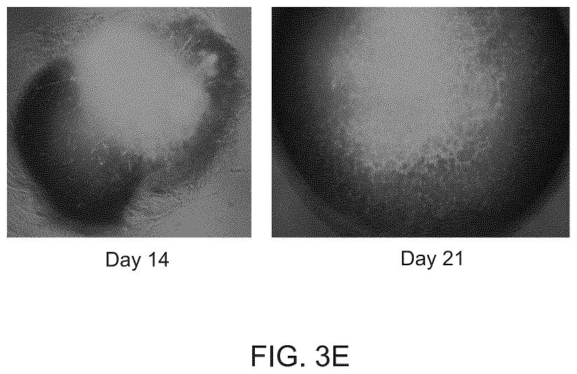

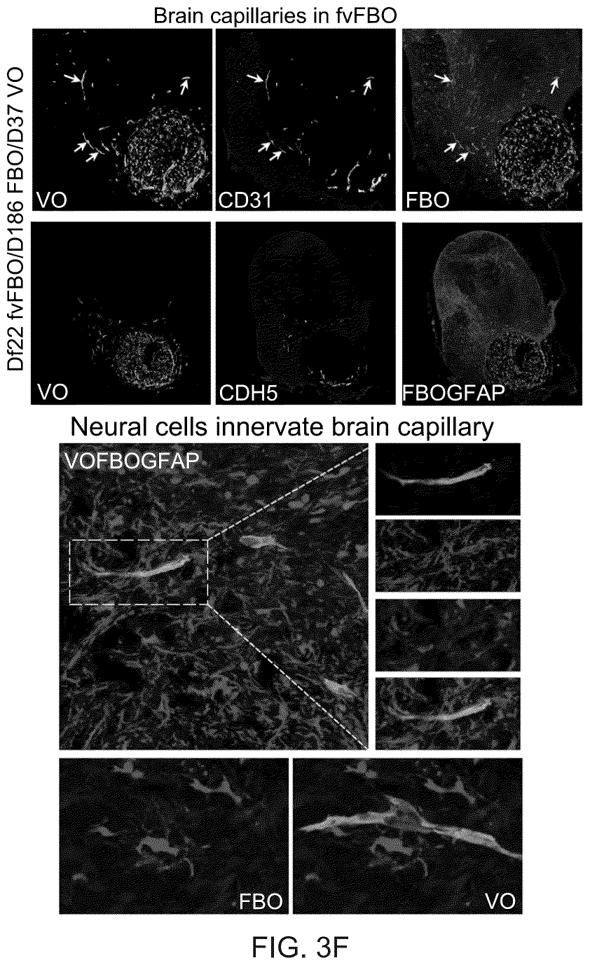

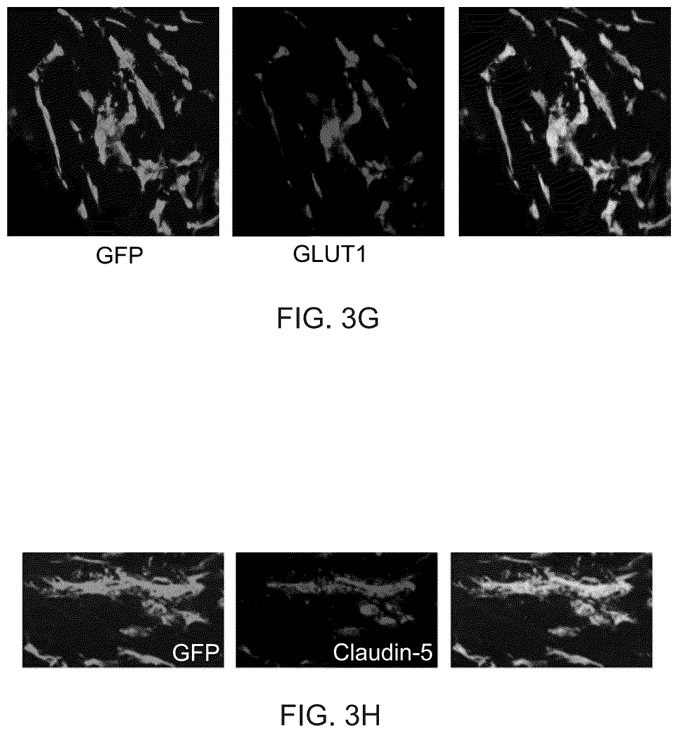

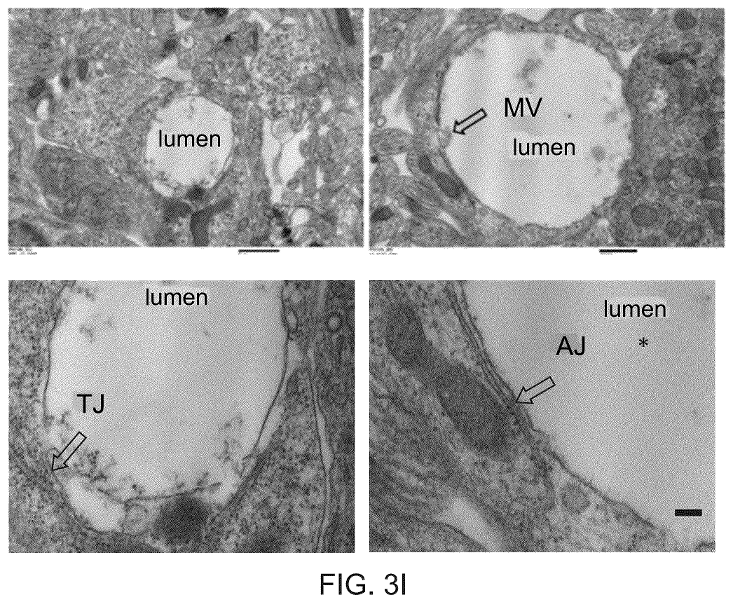

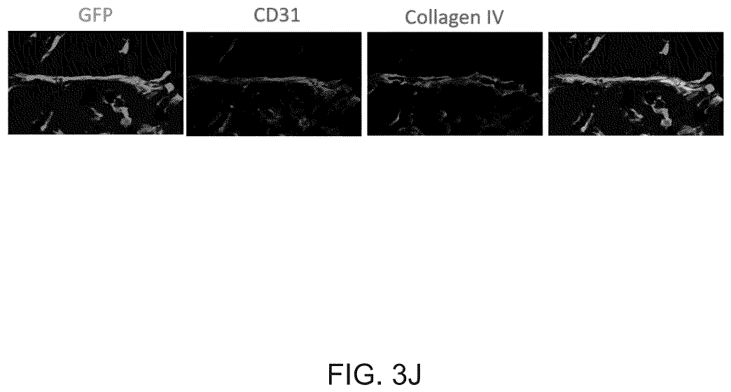

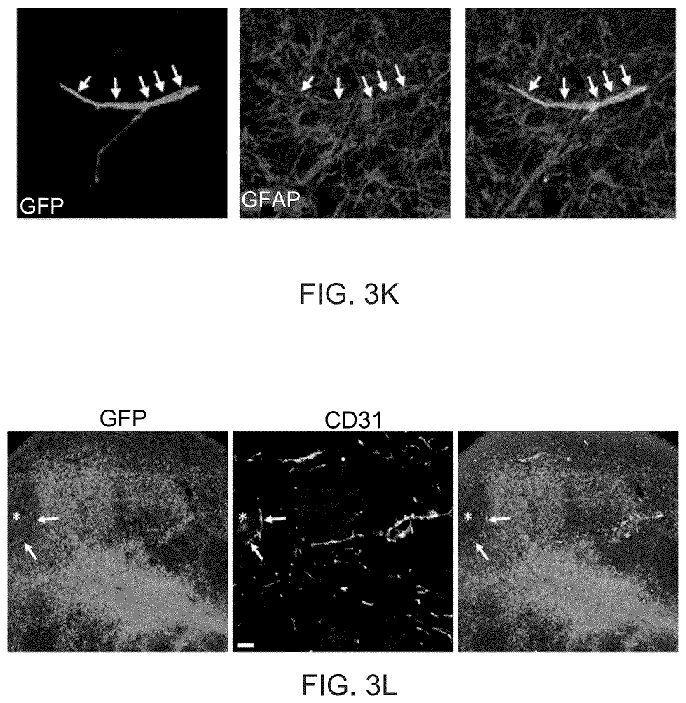

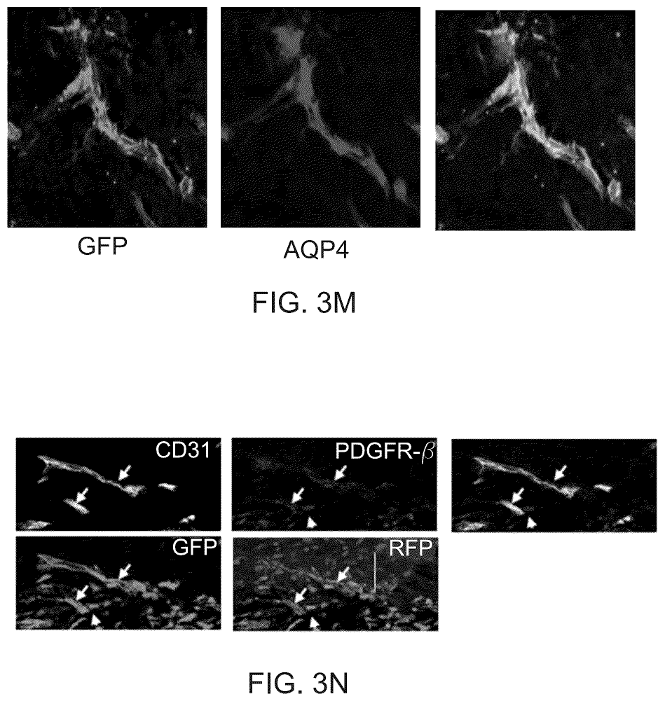

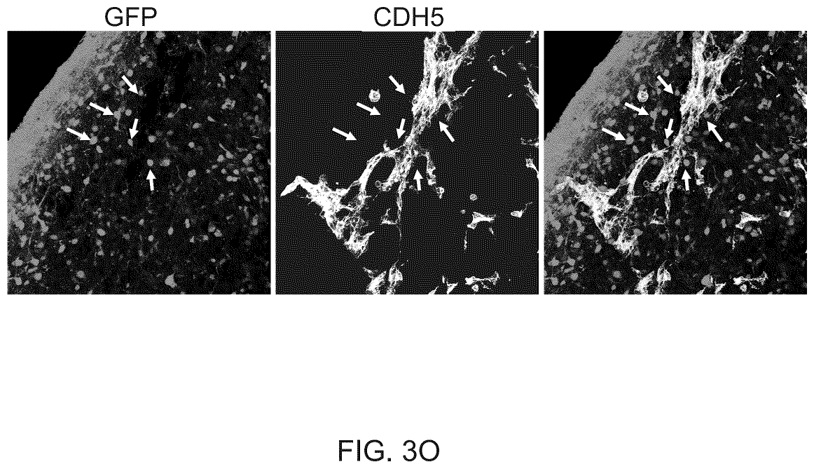

Blood vessel organoids and cortical organoids are produced according to methods provided herein (e.g., in the Examples), or otherwise generally known in the art. To generate vascularized cortical organoids modeling an intact blood-brain barrier, cortical organoids cultured to contain astrocytes were used. A schematic for an approach for forming fused vascularized cortical organoids is provided in FIGS. 3A, 3B and 3D.

A Matrigel mixture of 100 μL Matrigel and 60 μL of ice cold brain vascularization medium (a 50%/50% mixture of a) StemPro-34 medium with 15% FBS, 100 ng/mL VEGF, and 100 ng/ml FGF2 and b) F4 medium (neurobasal medium, 1× B27, 1× Glutamax, 1× NEAA, 1× 1× β-mercaptocthanol, 1× penicillin-streptomycin supplemented with 0.05 mM cAMP, 0.2 mM ascorbic acid, 20 ng/ml BDNF, and 20 ng/mL GDNF)) was prepared. This preparation volume is sufficient for making approximately 5 vascularized cortical organoids using 30 μL of the mixture for each.

A single blood vessel organoid and a single cortical organoid was placed in a 1.5 mL tube and any medium in the tube carried over from transferring the organoids was removed. In this process, it was ensured that the two organoids are in direct contact with each other. 30 μL of the Matrigel/brain vascularization medium mixture was added to the organoids, and the tube was placed in a 37° C. incubator to solidify the Matrigel mixture. The tube can be gently centrifuged prior to Matrigel solidification if needed to settle smaller sized organoids. Additional brain vascularization medium was added to the tube, and the tube was then incubated at 37° C. for a day. After a day, half of the liquid medium was changed with fresh brain vascularization medium. The tube should be opened for approximately 30-60 minutes each day in a sterile hood to allow for gas exchange. After another day, the Matrigel droplet was transferred to a low attachment 6 well plate containing brain vascularization medium and cultured for an additional 3 days. After the 3 days, the plate was incubated at 37° C. with low speed shaking (100 rpm) for 3 days. Subsequently, the plate was incubated at 37° C. with higher speed shaking (120 rpm). After a total of 20 days from the initial blood vessel organoid and cortical organoid contacting in Matrigel, the fused organoid was harvested for use.

In an alternative approach, a single blood vessel organoid and a single cortical organoid was placed onto a sterile surface (e.g., piece of sterile plastic). A droplet of the Matrigel/brain vascularization medium mixture was placed on the sterile surface, and the two organoids were manipulated to be in direct contact with each other in the center of the Matrigel/brain vascularization medium mixture. The sterile surface holding the organoids were placed in a 37° C. incubator to solidify the Matrigel mixture. Subsequently, the solidified Matrigel droplet containing the organoids was transferred to a suitable tissue culture plate containing brain vascularization medium and incubated at 37° C. for 4 days. After the 4 days, the plate was incubated at 37° C. with low speed shaking (100 rpm) for 3 days. Subsequently, the plate was incubated at 37° C. with higher speed shaking (120 rpm). After a total of 20 days from the initial blood vessel organoid and cortical organoid contacting in Matrigel, the fused organoid was harvested for use.

As shown in FIGS. 3D and 3E, the direct contact of blood vessel organoids and forebrain organoid led to their fusion, and infiltration of endothelial cells from the blood vessel organoids into the forebrain organoid. The formation of brain capillaries within the forebrain organoid was confirmed by detection of endothelial cell markers CD31 and cadherin 5 (CDH5) within the forebrain organoid, with neural cells closely associated with the endothelial cells. FIGS. 3K and 3N show the presence of GFAP-positive astrocytes and PDGFR-β-positive pericyte progenitor cells, which are additional cell types innately involved in the blood-brain barrier. Importantly, FIG. 3H shows the expression of claudin-5, which represents the tight junctions that are critical for blood-brain barrier function, and which has been observed to be absent or weakly present in prior models of the BBB.

Further by day 21, the majority of endothelial cells expressed BBB-specific markers such as glucose transporter 1 (Glut-1) and tight junction proteins such as Claudin-5 (FIGS. 3G-H) and (ZO-1 (not shown), indicating that the endothelial cells were differentiating towards a BBB-specific fate. Day 21 vascularized brain organoids immunostained for GFP, CD31, and Collagen IV shows that the brain endothelium (CD31) was covered by a continuous basement membrane, an important structure for regulating angiogenesis and maintaining the BBB, which was defined by the molecular marker Collagen IV (FIG. 3J). It is also noteworthy that astrocytic processes labeled by GFAP were well-aligned with endothelial tubes (FIG. 3K-L) and that human astrocytes extended their end-feet labeled by AQP-4 to wrap up the abluminal capillary surface (FIG. 3M). The newly form capillaries were not only ensheathed by astrocytic processes but also by human pericytes (stained for PDGFR-β, FIG. 3N), indicative of the resemblance of in vivo human BBB-like structure with endothelial cells of the capillary wall connected through tight junctions, astrocytic end-feet and pericytes ensheathing, as well as neuron innervation (FIG. 3C and FIG. 30).

FIG. 31 depicts electron micrographs of the fused vascularized forebrain organoid showing the presence of microvesicles (MV) protruding into the lumen of the brain capillary structure, tight junctions (TJ) and adherens junctions (AJ). This demonstrates that human brain microvascular endothelial cells (BMECs) formed capillaries through tight junctions.

### Example 4. Single Cell Transcriptomic Profiling of Fused Vascularized Cortical Organoids

The fused vascularized forebrain organoids were analyzed by single cell RNA transcriptomic sequencing. A total of 9342 cells were analyzed after quality control. Cells with mitochondrial gene ratios greater than 10% and less than 200 genes express were excluded. Clustering resolution was set to 0.5.

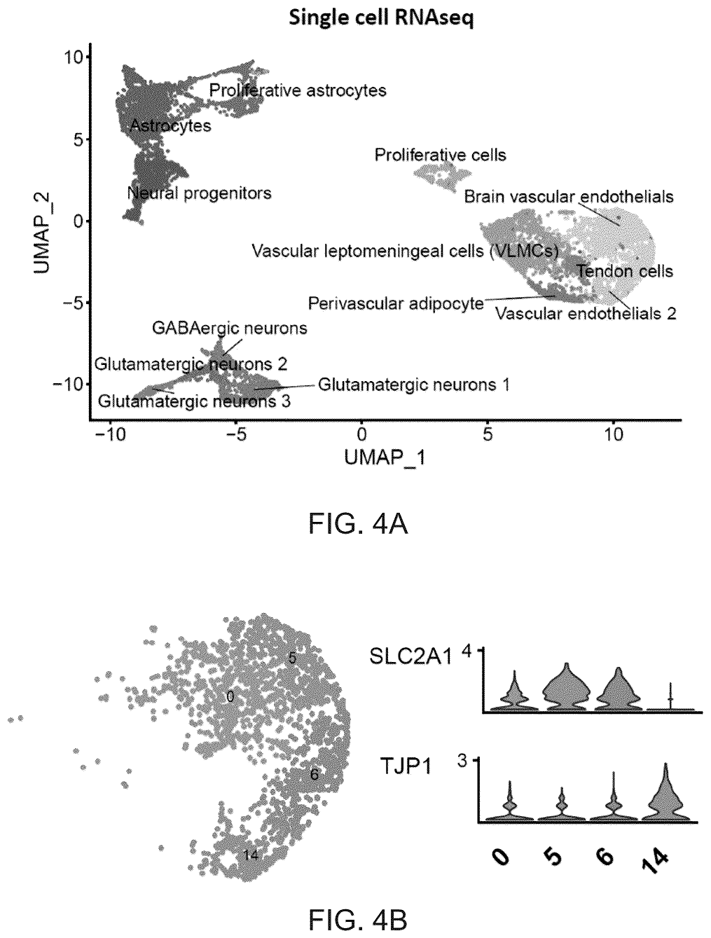

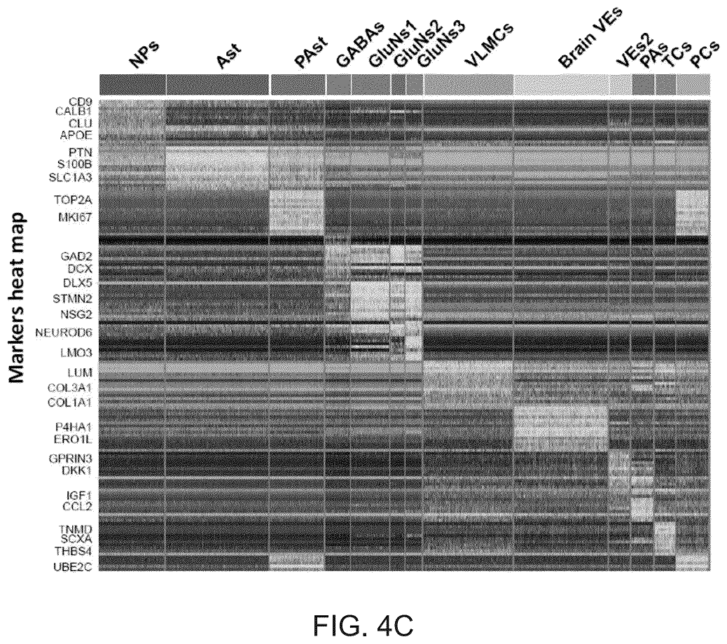

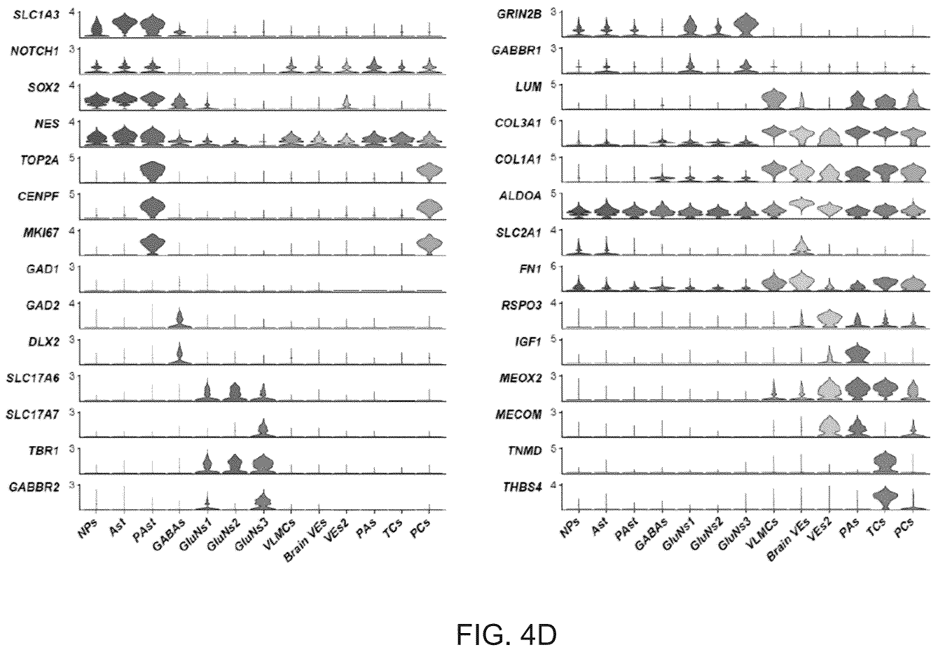

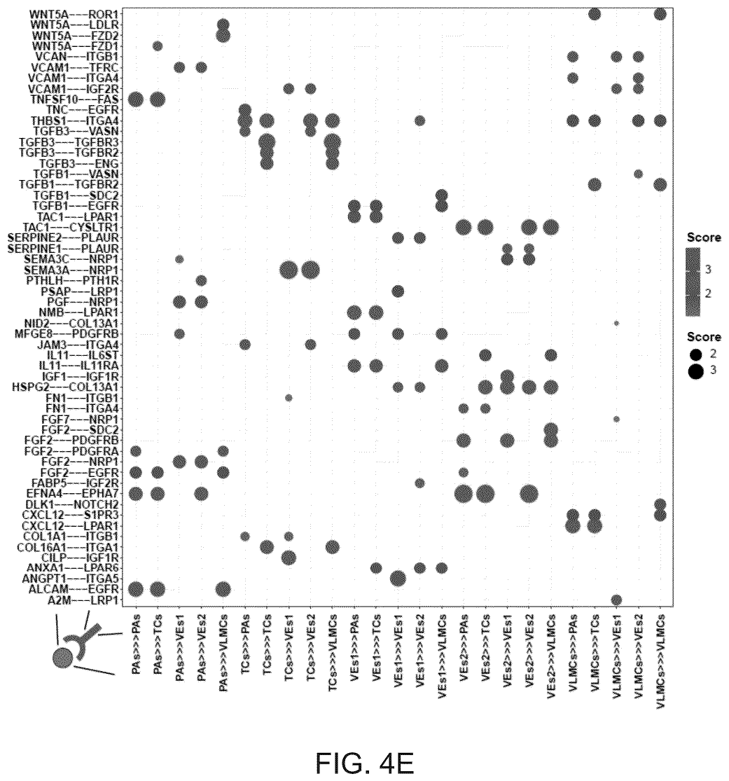

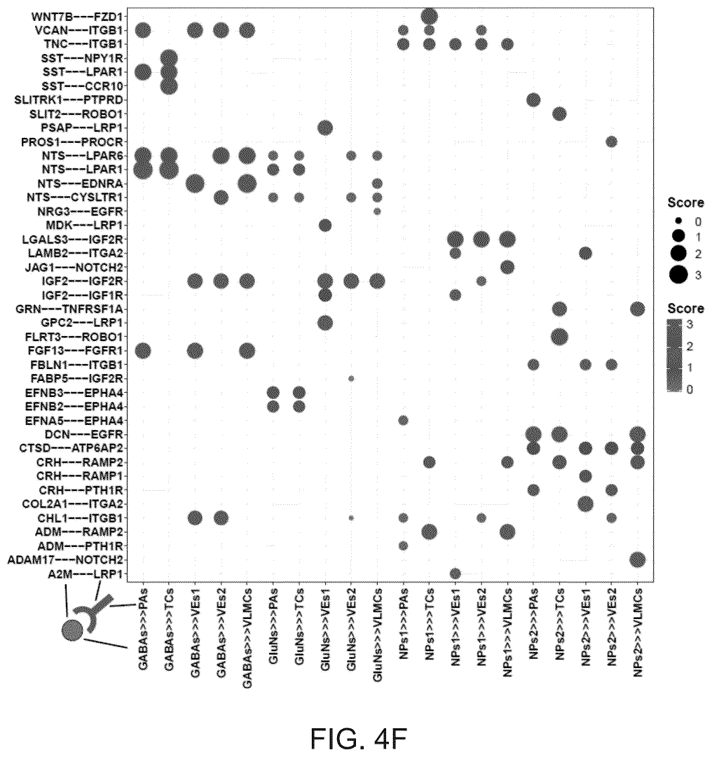

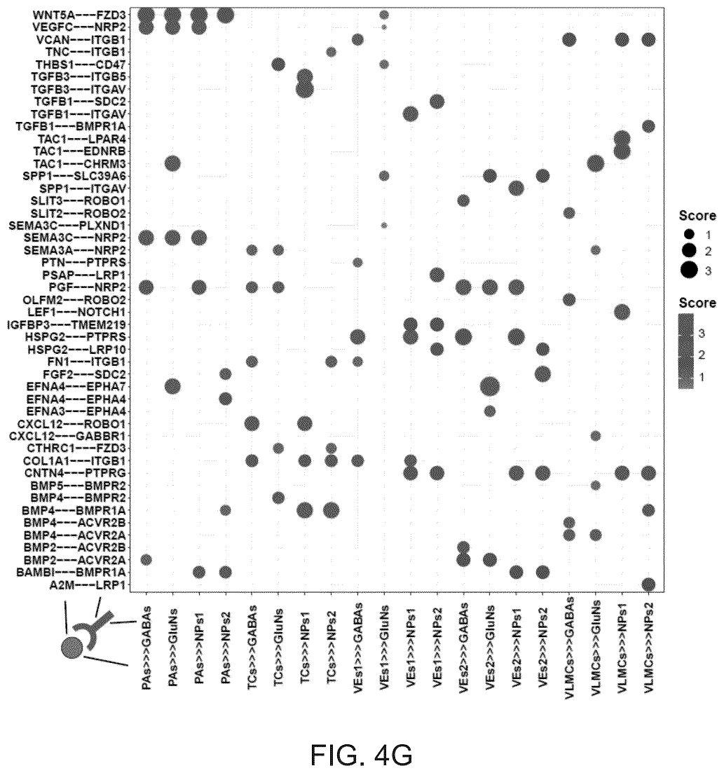

As shown in FIG. 4A, the vascularized organoids contain a multitude of cell types that are representative of the blood-brain barrier, including neurons, astrocytes, and endothelial cells. FIG. 4B shows that among the endothelial cell cluster, there exist sub-clusters, suggesting that the organoid contain diverse populations of cell types. FIGS. 4C and 4D show the relative expression of various cell markers in the different cell types identified in the single cell RNA sequencing. FIG. 4E-4G show maps of interactions between different known protein receptors and ligands expressed by cells associated with communication from 1) vascular cell types to vascular cell types, 2) neural cell types to vascular cell types, and 3) vascular cell types to neural cell types, respectively.

### Example 5: Additional Single-Cell Transcriptomic Profiling of Vascularized Brain Organoids

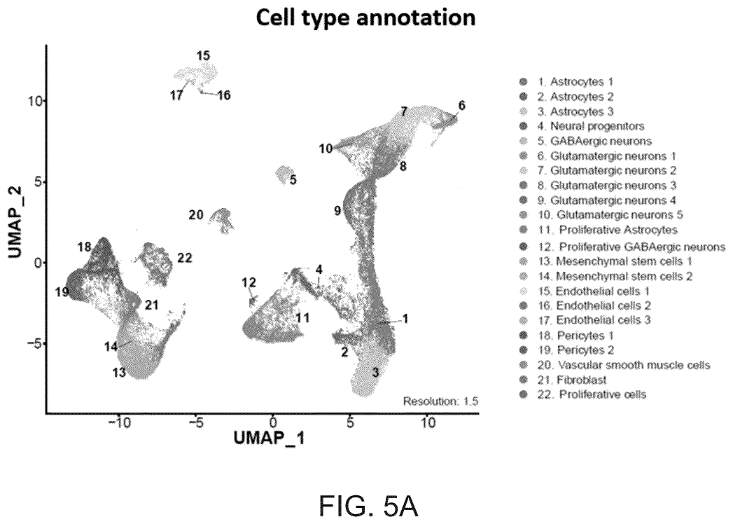

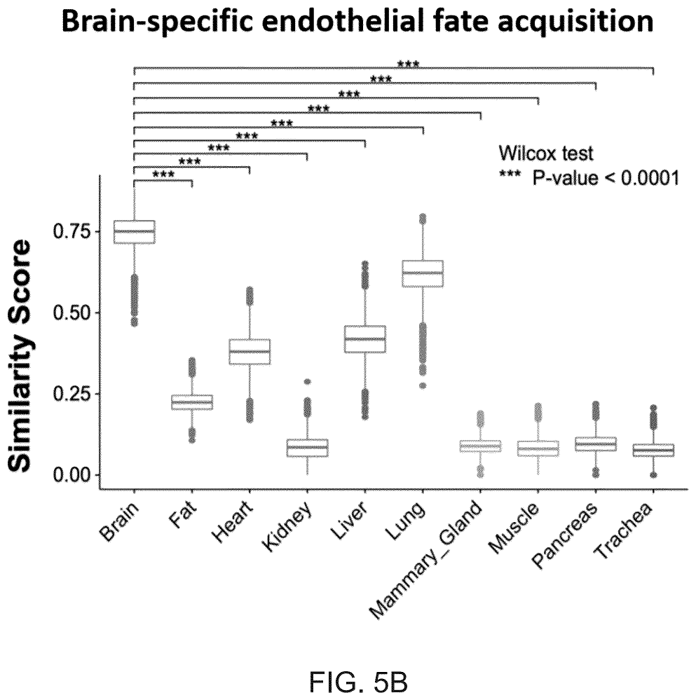

Additional single-cell RNA sequencing (scRNA-seq) was performed on Day 30 vascularized brain organoids derived from H9 embryonic stem cells with three replicate cultures to comprehensively decipher the cell populations present in vascularized brain organoids at the transcriptomic level. The sequencing data was aligned and quantified using Cell Ranger (10x Genomics) to obtain raw count data. The R package Seurat (version 4) was used to normalize the raw count data and DoubletFinder was applied to remove doublet cells in the scRNA-seq data. After doublet cell removal, a total of 28,062 cells were extracted for further analysis (n=three cultures). FindClusters was then applied to identify differentially expressed gene markers for each cell cluster. FIG. 5A shows clusters of excitatory neurons, inhibitory neurons, neural progenitors (NPs), astrocytes, endothelial cells (ECs), pericytes, mesenchymal stem cells (MSCs), smooth muscle cells (SMCs), and fibroblasts identified by gene expression markers. The presence of these cell types suggests that vascularized brain organoids can resemble a complete neurovascular unit. The single-cell transcriptomics analysis showed a similar cell population between biological replicas, indicative of the reliability of the protocol. Gene expression data from endothelial cells in vascularized brain organoids was compared with gene expression data from organ-specific endothelial cells generated by the Tabula Muris Consortium. FIG. 5B shows that endothelial cells in vascularized brain organoids presented an identical gene expression pattern with brain microvascular endothelial cells (BMECs) but not with other organ-specific endothelial cells, indicative of the endothelial acquisition of brain-specific transcriptomic signatures in vascularized brain organoids.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Blanchard et al. Reconstruction of the human blood-brain barrier in vitro reveals a pathogenic mechanism of APOE4 in pericytes. Nature Medicine (2020) 26 (6): 952-963.
Cakir et al. Engineering of human brain organoids with a functional vascular-like system. Nature Methods (2019) 16 (11): 1169-1175.
Chandrasekaran et al. Astrocyte Differentiation of Human Pluripotent Stem Cells: New Tools for Neurological Disorder Research. Front Cell. Neurosci. (2016) 10:215.
Daviaud et al. Vascularization and Engraftment of Transplanted Human Cerebral Organoids in Mouse Cortex. eNeuro (2018) 5 (6): ENEURO.0219-18.
Ham et al. Blood vessel formation in cerebral organoids formed from human embryonic stem cells. Biochem Biophys. Res. Commun. (2020) 521 (1): 84-90.
Huang et al. Generation of hypothalamic arcuate organoids from human induced pluripotent stem cells. Cell Stem Cell (2021) 28 (9): 1657-1670.
Jacob et al. Human Pluripotent Stem Cell-Derived Neural Cells and Brain Organoids Reveal SARS-COV-2 Neurotropism Predominates in Choroid Plexus Epithelium. Cell Stem Cell. (2020) 27 (6): 937-950.
Lippmann et al. Human Blood-Brain Barrier Endothelial Cells Derived from Pluripotent Stem Cells. Nat. Biotechnol. (2012) 30 (8): 783-791.
Mansour et al. An in vivo model of functional and vascularized human brain organoids Nature Biotechnology (2018) 36:432-441.
Perriot et al. Differentiation of functional astrocytes from human-induced pluripotent stem cells in chemically defined media. STAR Protoc. (2021) 2 (4): 100902.
Perriot et al. Human Induced Pluripotent Stem Cell-Derived Astrocytes Are Differentially Activated by Multiple Sclerosis-Associated Cytokines. Stem Cell Reports. (2018) 11 (5): 1199-1210.
Qian et al. Brain-Region-Specific Organoids Using Mini-bioreactors for Modeling ZIKV Exposure. Cell (2016) 165 (5): 1238-1254.
Qian et al. Generation of human brain region-specific organoids using a miniaturized spinning bioreactor. Nature Protocols (2018) 13 (3): 565-580.
Shi et al. Vascularized human cortical organoids (vOrganoids) model cortical development in vivo. PLoS Biol. (2020) 18 (5): e3000705.
Shin et al. Blood-Brain Barrier Dysfunction in a 3D In Vitro Model of Alzheimer's Disease. Adv. Sci. (Weinh). (2019) 6 (20): 1900962.
Song et al. Assembly of Human Stem Cell-Derived Cortical Spheroids and Vascular Spheroids to Model 3-D Brain-like Tissues. Scientific Reports 9:5977.
TCW et al. An Efficient Platform for Astrocyte Differentiation from Human Induced Pluripotent Stem Cells. Stem Cell Reports. (2017) 9 (2): 600-614.
Wimmer et al. Generation of blood vessel organoids from human pluripotent stem cells. Nature Protocols (2019) 14 (11): 3082-3100.
Wimmer et al. Human blood vessel organoids as a model of diabetic vasculopathy. Nature (2019) 565 (7740): 505-510.

What is claimed is:

1. A method for producing a vascularized brain organoid, comprising:
contacting a blood vessel organoid and a brain organoid; and
culturing the blood vessel organoid and the brain organoid for a period of time until the blood vessel organoid and the brain organoid fuse together and blood vessels of the blood vessel organoid infiltrate the brain organoid;
wherein neurons of the brain organoid innervate the blood vessels of the blood vessel organoid that have infiltrated the organoid;
thereby forming the vascularized brain organoid;
wherein the vascularized brain organoid comprises a blood-brain barrier that is formed between the brain organoid and all or a portion of the blood vessels of the blood vessel organoid that have infiltrated the brain organoid;
wherein the blood-brain barrier comprises endothelial cells linked with tight junctions, astrocytes, and pericytes.

2. The method of claim 1, wherein the endothelial cells express CD31, GLUT-1 and PDGFR-β; the tight junctions comprise claudin-5, ZO-1 and cadherin 5; the astrocytes express S100B, GFAP, and AQP4; and the pericytes express PDGFR-β, αSMA and NG2.

3. The method of claim 1 or 2, wherein the endothelial cells form a continuous basement membrane and express collagen IV.

4. The method of any of claims 1-3, wherein the vascularized brain organoid comprises cells selected from the group consisting of neural progenitors, proliferative astrocytes, GABAergic neurons, glutamatergic neurons, proliferative cells, brain vascular endothelial cells, vascular leptomeningeal cells, perivascular adipocytes, and tendon cells.

5. The method of any of claims 1-3, wherein the vascularized brain organoid comprises cells selected from the group consisting of neural progenitor cells, GABAergic neurons, glutamatergic neurons, proliferative astrocytes, proliferative GABAergic neurons, mesenchymal stem cells, endothelial cells, pericytes, vascular smooth muscle cells, fibroblast, and proliferative cells.

6. The method of claim 4 or 5, wherein the cells of the vascular brain organoid are identified by cell-type specific gene expression markers.

7. The method of any of claims 1-6, wherein the blood vessels comprise capillaries.

8. The method of claim 7, wherein the capillaries are ensheathed by pericytes and end-feet of the astrocytes.

9. The method of any of claims 1-8, wherein the brain organoid is a forebrain organoid, a midbrain organoid, a hypothalamus organoid, a hippocampus organoid, a spinal cord organoid, or a striatal brain organoid.

10. The method of any one of claims 1-9, wherein the blood vessel organoid and the brain organoid are contacted and/or cultured in a basement membrane matrix or component thereof, optionally Matrigel.

11. The method of any one of claims 1-10, wherein the blood vessel organoid and the brain organoid are cultured for a period of time that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days.

12. The method of any one of claims 1-11, wherein the blood vessel organoid and the brain organoid are cultured with agitation, optionally shaking, for at least a portion of the period of time.

13. The method of any one of claims 1-12, wherein the blood vessel organoid and the brain organoid are cultured:
1) without agitation for 1, 2, 3, 4, 5, 6, 7, 8, 9, 10, 11, 12, 13, or 14 days, or any number of days within a range defined by any two of the aforementioned days; and subsequently
2) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned days;
optionally wherein the agitation comprises shaking.

14. The method of any one of claims 1-13, wherein the blood vessel organoid and the brain organoid are cultured in a medium that promotes neuronal growth and/or vascular growth.

15. The method of any one of claims 1-14, wherein the blood vessel organoid and the brain organoid are cultured in a medium that comprises growth factors that promote neuronal growth and/or growth factors that promote vascular growth.

16. The method of claim 15, wherein the growth factors that promote neuronal growth comprise a cAMP pathway activator, ascorbic acid, BDNF, GDNF, or any combination thereof.

17. The method of claim 15 or 16, wherein the growth factors that promote vascular growth comprise growth serum, a VEGF pathway activator, an FGF pathway activator, or any combination thereof.

18. The method of any one of claims 1-17, wherein culturing the blood vessel organoid and the brain organoid comprises:
a) culturing the blood vessel organoid and the brain organoid without agitation for 4, 5, 6, 7, 8, 9, or 10 days, optionally 7 days; and
b) culturing the organoids of step a) with agitation for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days;
wherein the organoids of step a) and step b) are cultured in a medium comprising growth factors that promote neuronal growth and/or growth factors that promote vascular growth;
optionally wherein the agitation comprises shaking;
optionally wherein the growth factors that promote neuronal growth comprise a cAMP pathway activator, ascorbic acid, BDNF, GDNF, or any combination thereof;
optionally wherein the growth factors that promote vascular growth comprise growth serum, a VEGF pathway activator, an FGF pathway activator, or any combination thereof;
optionally wherein the blood vessel organoid and the brain organoid are cultured in a basement membrane matrix or component thereof, optionally Matrigel.

19. The method of any one of claims 16-18, wherein the CAMP pathway activator is cAMP.

20. The method of any one of claims 16-19, wherein the CAMP pathway activator is provided at a concentration of, or of about, 10, 20, 30, 40, 50, 60, 70, 80, 90, 100, 110, 120, 130, 140, or 150 μM, or any concentration within a range defined by any two of the aforementioned concentrations, optionally 50 μM or about 50 μM.

21. The method of any one of claims 16-20, wherein the ascorbic acid is provided at a concentration of, or of about, 50, 60, 70, 80, 90, 100, 110, 120, 130, 140, 150, 160, 170, 180, 190, 200, 210, 220, 230, 240, 250, 260, 270, 280, 290, or 300 μM, or any concentration within a range defined by any two of the aforementioned concentrations, optionally 200 μM or about 200 μM.

22. The method of any one of claims 16-21, wherein the BDNF is provided at a concentration of, or of about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, optionally 20 ng/ml or about 20 ng/mL.

23. The method of any one of claims 16-22, wherein the GDNF is provided at a concentration of, or of about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ng/mL, or any concentration within a range defined by any two of the aforementioned concentrations, optionally 20 ng/ml or about 20 ng/mL.

24. The method of any one of claims 17-23, wherein the growth serum is fetal bovine serum (FBS).

25. The method of any one of claims 17-24, wherein the growth serum is provided at a concentration of or of about, 0.5%, 1%, 2%, 3%, 4%, 5%, 6%, 7%, 8%, 9%, 10%, 11%, 12%, 13%, 14%, 15%, 16%, 17%, 18%, 19%, or 20%, or any concentration within a range defined by any two of the aforementioned concentrations, optionally 15% or about 15%.

26. The method of any one of claims 17-25, wherein the VEGF pathway activator is VEGF.

27. The method of any one of claims 17-26, wherein the VEGF pathway activator is provided at a concentration of, or of about, 10, 20, 30, 40, 50, 60, 70, 80, 90, 100, 110, 120, 130, 140, or 150 ng/mL, or any concentration within a range defined by any two of the aforementioned concentrations, optionally 100 ng/ml or about 100 ng/ml.

28. The method of any one of claims 17-27, wherein the FGF pathway activator is FGF2.

29. The method of any one of claims 17-28, wherein the FGF pathway activator is provided at a concentration of, or of about, 10, 20, 30, 40, 50, 60, 70, 80, 90, 100, 110, 120, 130, 140, or 150 ng/mL, or any concentration within a range defined by any two of the aforementioned concentrations, optionally 100 ng/mL or about 100 ng/mL.

30. The method of any one of claims 1-29, wherein the blood vessel organoid and/or the brain organoid are derived from pluripotent stem cells, optionally embryonic stem cells or induced pluripotent stem cells.

31. The method of any one of claims 1-30, wherein the blood vessel organoid has been produced according to a method comprising:
contacting an angiogenic sprout with an FGF pathway activator, a VEGF pathway activator, optionally a Wnt pathway activator, and optionally a growth serum, for a first period of time;
thereby forming the blood vessel organoid.

32. The method of claim 31, wherein the first period of time is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any number of days within a range defined by any two of the aforementioned number of days, optionally 5 days or at least 5 days.

33. The method of claim 31 or 32, wherein the angiogenic sprout has been produced according to a method comprising:
a) contacting pluripotent stem cells with a Wnt pathway activator and a BMP pathway activator for a second period of time to form vascular lineage cells; and
b) contacting the vascular lineage cells with a VEGF pathway activator and a second cAMP pathway activator for a third period of time;
thereby forming the angiogenic sprout.

34. The method of claim 33, wherein the second period of time is 1, 2, 3, 4, or 5 days, or any number of days within a range defined by any two of the aforementioned number of days, optionally 3 days.

35. The method of claim 33 or 34, wherein the third period of time is 1, 2, 3, or 4 days, or any number of days within a range defined by any two of the aforementioned number of days, optionally 2 days.

36. The method of any one of claims 33-35, wherein the BMP pathway activator is BMP4.

37. The method of any one of claims 33-36, wherein the BMP pathway activator is provided at a concentration of, or of about, 10, 15, 20, 25, 30, 35, 40, 45, 50, 55, 60, 65, 70, 75, 80, 85, 90, 95, or 100 ng/mL, or any concentration within a range defined by any two of the aforementioned concentrations, optionally 30 ng/mL or about 30 ng/mL.

38. The method of any one of claims 31-37, wherein the Wnt pathway activator is CHIR99021.

39. The method of any one of claims 31-38, wherein the Wnt pathway activator is provided at a concentration of, or of about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, optionally 4 μM or about 4 μM, or optionally 12 μM or about 12 μM.

40. The method of any one of claims 33-39, wherein the second cAMP pathway activator is forskolin.

41. The method of any one of claims 33-40, wherein the second cAMP pathway activator is provided at a concentration of, or of about, 0.5, 1, 1.5, 2, 2.5, 3, 3.5, or 4 μM, or any concentration within a range defined by any two of the aforementioned concentrations, optionally 2 μM or about 2 μM.

42. The method of any one of claims 31-41, wherein the blood vessel organoid differs from a blood vessel organoid that has been produced without contacting the angiogenic sprout with the Wnt pathway activator by having increased expression of blood-brain barrier-specific endothelial markers, optionally GLUT-1 and ZO-1.

43. The method of any one of claims 1-42, wherein the brain organoid has been contacted with LIF and growth serum to induce astrocyte formation in the brain organoid.

44. The method of any one of claims 1-43, wherein the brain organoid has been produced according to a method comprising:
a) contacting pluripotent stem cells with a BMP pathway inhibitor, a TGF-beta pathway inhibitor, and a Wnt pathway inhibitor for a first period of time to form neuroectoderm cells;
b) contacting the neuroectoderm cells of step a) with a second TGF-beta pathway inhibitor, and a Wnt pathway activator for a second period of time to form neuroepithelium cells;
c) contacting the neuroepithelium cells of step b) with insulin for a third period of time to form cerebral tissue organoids; and
d) contacting the cerebral tissue organoid of step c) with GDNF, BDNF, ascorbic acid, and a cAMP pathway activator for a fourth period of time to form the brain organoid;
optionally wherein the cerebral tissue organoid is further contacted with LIF and growth serum for a portion of the fourth period of time to induce astrocyte proliferation in the brain organoid.

45. The method of claim 44, wherein the first period of time is 1, 2, 3, 4, 5, 6, 7, 8, 9, 10, 11, 12, 13, or 14 days, or any number of days within a range defined by any two of the aforementioned number of days, optionally 7 days.

46. The method of claim 44 or 45, wherein the second period of time is 1, 2, 3, 4, 5, 6, 7, 8, 9, 10, 11, 12, 13, or 14 days, or any number of days within a range defined by any two of the aforementioned number of days, optionally 7 days.

47. The method of any one of claims 44-46, wherein the third period of time is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days, optionally 56 days.

48. The method of any one of claims 44-47, wherein the fourth period of time is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days, or any number of days within a range defined by any two of the aforementioned number of days.

49. The method of any one of claims 44-48, wherein the portion of the fourth period of time is 7, 8, 9, 10, 11, 12, 13, 14, 15, 16, 17, 17, 18, 19, 20, or 21 days, or any number of days within a range defined by any two of the aforementioned number of days, optionally 14 days, optionally wherein the portion of the fourth period of time is at the beginning of the fourth period of time.

50. The method of any of claims 44-49, wherein the BMP pathway inhibitor is LDN-193189.

51. The method of any one of claims 44-50, wherein the BMP pathway inhibitor is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

52. The method of any one of claims 44-51, wherein the TGF-beta pathway inhibitor and the second TGF-beta pathway inhibitor is the same or different.

53. The method of any one of claims 44-52, wherein the TGF-beta pathway inhibitor is A83-01.

54. The method of any one of claims 44-53, wherein the TGF-beta pathway inhibitor is provided at a concentration of, or of about, 0.5, 1, 1.5, 2, 2.5, 3, 3.5, or 4 μM, or any concentration within a range defined by any two of the aforementioned concentrations, optionally 2 μM or about 2 μM.

55. The method of any one of claims 44-54, wherein the second TGF-beta pathway inhibitor is SB-431542.

56. The method of any one of claims 44-55, wherein the second TGF-beta pathway inhibitor is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

57. The method of any one of claims 44-56, wherein the Wnt pathway inhibitor is IWR-1.

58. The method of any one of claims 44-57, wherein the Wnt pathway inhibitor is provided at a concentration of, or of about, 0.5, 1, 1.5, 2, 2.5, 3, 3.5, 4, 4.5, or 5 μM, or any concentration within a range defined by any two of the aforementioned concentrations, optionally 3 μM or about 3 μM.

59. The method of any one of claims 44-58, wherein the Wnt pathway activator is CHIR99021.

60. The method of any one of claims 44-59, wherein the Wnt pathway activator is provided at a concentration of, or of about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 μM, or any concentration within a range defined by any two of the aforementioned concentrations, optionally 1 μM or about 1 μM.

61. The method of any one of claims 44-60, wherein the insulin is provided at a concentration of, or of about, 0.5, 1, 1.5, 2, 2.5, 3, 3.5, 4, 4.5, or 5 μg/mL, or any concentration within a range defined by any two of the aforementioned concentrations, optionally 2.5 μg/mL or about 2.5 μg/mL.

62. The method of any one of claims 44-61, wherein the LIF is provided at a concentration of, or of about, 1, 2, 3, 4, 5, 6, 7, 8, 9, 10, 11, 12, 13, 14, 15, 16, 17, 18, 19, or 20 mg/mL, optionally 10 mg/mL or about 10 mg/mL.

63. The method of any one of claims 44-62, wherein the brain organoid comprises astrocytes that express S100B, GFAP, and AQP4.

64. The method of any one of claims 1-63, wherein the blood vessel organoid and/or the brain organoid are human.

65. The method of any one of claims 1-64, wherein the blood vessel organoid and/or the brain organoid have been derived from a subject, optionally a human subject.

66. The method of claim 65, wherein the subject comprises a cerebrovascular disease or a disease associated with blood-brain barrier dysfunction, optionally wherein the cerebrovascular disease or disease associated with blood-brain barrier dysfunction comprises cerebral cavernous malformation, Alzheimer's disease, or amyotrophic lateral sclerosis.

67. The vascularized brain organoid produced by the method of any one of claims 1-66.

68. A vascularized brain organoid comprising endothelial cells linked with tight junctions, astrocytes, and pericytes.

69. The vascularized brain organoid of claim 68, wherein the endothelial cells express CD31, GLUT-1 and PDGFR-β; the tight junctions comprise claudin-5, ZO-1 and cadherin 5; the astrocytes express S100B, GFAP, and AQP4; and the pericytes express PDGFR-β, αSMA and NG2

70. The vascularized brain organoid of claim 68 or 69, wherein the endothelial cells form a continuous basement membrane and express collagen IV.

71. The vascularized brain organoid of any of claims 68-70 comprising cells selected from the group consisting of neural progenitors, proliferative astrocytes, GABAergic neurons, glutamatergic neurons, proliferative cells, brain vascular endothelial cells, vascular leptomeningeal cells, perivascular adipocytes, and tendon cells.

72. The vascularized brain organoid of any of claims 68-71 comprising cells selected from the group consisting of neural progenitor cells, GABAergic neurons, glutamatergic neurons, proliferative astrocytes, proliferative GABAergic neurons, mesenchymal stem cells, endothelial cells, pericytes, vascular smooth muscle cells, fibroblast, and proliferative cells.

73. The vascularized brain organoid of claim 71 or 72, wherein the cells are identified by cell-type specific gene expression markers.

74. The vascularized brain organoid of any of claims 68-73, wherein the blood vessels comprise capillaries.

75. The vascularized brain organoid of any of claims 68-74, wherein the capillaries are ensheathed by pericytes and end-feet of the astrocytes.

76. A method of treating a cerebrovascular disease or a disease associated with blood-brain barrier dysfunction in a subject in need thereof, comprising administering the vascularized brain organoid of any of claims 67-75, or a portion or fragment thereof, to the subject.

77. A method of screening, comprising contacting the vascularized brain organoid of any of claims 67-75, or portions thereof, with a candidate compound or composition, and assessing the effects of the candidate compound or composition on the vascularized brain organoid or portions thereof.

78. The method of claim 77, wherein the effects comprises transport of the candidate compound or composition across the blood-brain barrier of the organoid or portions thereof.

79. The method of claim 77 or 78, wherein the vascularized brain organoid is a model for a cerebrovascular disease or a disease associated with blood-brain barrier dysfunction, and assessing the effects of the candidate compound or composition on the vascularized organoid comprises assessing the effects of the candidate compound or composition on the cerebrovascular disease or the disease associated with blood-brain barrier dysfunction.

80. The method of any one of claims 77-79, wherein the vascularized brain organoid has been produced from cells derived from a subject, optionally wherein the cells derived from the subject are induced pluripotent stem cells.

81. The method of claim 80, wherein the subject has or is disposed to develop the cerebrovascular disease or the disease associated with blood-brain barrier dysfunction.
